SEQUENCE LISTING (15 sequences)

SEQ ID NO: 1 - DNA, 20 bp, Artificial Sequence
  antisense to c-myc
acgttgaggggcatcgtcgc

SEQ ID NO: 2 - DNA, 20 bp, Artificial Sequence
  c-myc antisense scramble control
actgtgagggcgatcgctgc

SEQ ID NO: 3 - DNA, 21 bp, Artificial Sequence
  c-myc antisense control
catcaccaggattggacatgg

SEQ ID NO: 4 - DNA, 22 bp, Artificial Sequence
  c-myc antisense control
caagctgcatttgcatagccat

SEQ ID NO: 5 - DNA, 21 bp, Artificial Sequence
  antisense to c-myc
gmtmmmtmtgtmtmtmgmtgg

SEQ ID NO: 6 - DNA, 20 bp, Artificial Sequence
  antisense to c-myc
mmgmmmgmtmgmtmmmtmtg

SEQ ID NO: 7 - DNA, 30 bp, Artificial Sequence
  antisense to c-myc
ggcaucgucgugacugucggguuuuccacc

SEQ ID NO: 8 - DNA, 30 bp, Artificial Sequence
  antisense to c-myc
ggggcaucgucgugacugucuguuggaggg

SEQ ID NO: 9 - DNA, 21 bp, Artificial Sequence
  antisense to c-myc
cgucgugacugucuguuggag

SEQ ID NO: 10 - DNA, 22 bp, Artificial Sequence
  antisense to c-myc
cgtcgtgactgtctgttggagg

SEQ ID NO: 11 - DNA, 28 bp, Artificial Sequence
  antisense to c-myc
ggcaucgucgcgggaggcugcuggagcg

SEQ ID NO: 12 - DNA, 28 bp, Artificial Sequence
  antisense to c-myc
ccgcgacauaggacggagagcagagccc

SEQ ID NO: 13 - DNA, 12 bp, Artificial Sequence
  antisense to c-myc
ttgaggggcatc

SEQ ID NO: 14 - DNA, 15 bp, Artificial Sequence
  probe
gaggggcatcgtcgc

SEQ ID NO: 15 - DNA, 20 bp, Artificial Sequence
  probe
gcgacgatgcccctcaacgt

(19) United States
(12) Patent Application Publication
(10) Pub. No.: US 2003/0087861 A1
(43) Pub. Date: May 8, 2003

(54) Combined approach to treatment of cancer using a c-myc antisense oligomer

(76) Inventor: Patrick L. Iversen, Corvallis, OR (US)

Correspondence Address: PERKINS COIE LLP, P.O. BOX 2168, MENLO PARK, CA, 94026, US

(21) Appl. No.: 10/151,008

(22) Filed: May 17, 2002

Related U.S. Application Data
(60) Provisional application No. 60/291,727, filed on May 17, 2001.

(51) Int. Cl. A61K 48/00; C07H 21/04

(52) U.S. Cl. 514/44; 536/23.1

(57) ABSTRACT

Improved therapeutic methods for treatment of cancer by a combination treatment regimen that includes an oligomer to c-myc and a standard chemotherapeutic agent are provided.

This patent application claims priority to co-pending U.S. Provisional Application Serial No. 60/291,727, filed May 17, 2001, incorporated herein in its entirety by reference.

### FIELD OF THE INVENTION

The invention relates to methods for in vivo immunotherapy of cancer by administering an oligomer antisense to c-myc together with the administration of a traditional cancer chemotherapeutic agent.

### REFERENCES

Agrawal, S. et al., Proc. Natl. Acad. Sci. USA 87(4):1401-5, 1990.

Akhtar, S., et al., Nucleic Acids Res. 19(20):5551-9, 1991.

Amati B et al., Front Biosci 3:D250-68, 1998.

Anderson, C. M., et al., J Neurochem. 73(2):867-73, 1999.

Bennett et al., Circulation 92(7):1981-93, 1995.

Ben-Yosef et al., Oncogene, 17(2):165-71. 1998.

Bertram J S and Przemyslaw Cancer Letters 11:63-73, 1980.

Bieche I et al., Cancer Res 59(12):2759-65, 1999.

Blumenreich et al., Cancer. 55(5):1118-22, 1985.

Bonham, M. A. et al., Nucleic Acids Res. 23(7):1197-1203, 1995.

Boudvillain, M. et al., Biochemistry 36(10):2925-31, 1997.

Byhardt R W, Int J Radiat Oncol Biol Phys. 31(2):431-3,1995.

Dang C V et al., Exp. Cell Res. 253, 63-77,1999.

D'Cruz C M et al., Nature Med 7(2):235-39, 2001.

Denhardt, D T The Molecular Basis of Cell Cycle and Growth Control, G. S. Stein et al., Eds. pp. 225-304. 1999 Wiley-Liss, Inc.

Cohen et al., Antisense Res. & Dev. 2:191, 1991.

Coulis C M et al., Mol Pharmacol 57(3):485-94, 2000.

Forastiere et al., J Clin Oncol. 19(4):1088-95, 2001.

Gandara D R et al., Semin Oncol 18(1 Suppl 3):49-55, 1991.

Gandarillas A and Watt F M, Genes Dev 11 (21):2869-2882,1997.

Gauwerky C E and Croce C M, Semin Cancer Biol. 4(6):333-40, 1993.

Gee, J. E. et al., Antisense & Nucleic Acid Drug Dev. 8:103-111, 1998.

Ghosh, C et al., Meth. Enzym. 313:135-143.1999.

Giles G V et al., Antisense Nucleic Acid Drug Dev. 9:213-220, 1999.

Giles, R. V. et al., Anticancer Drug Des. 8(1):33-51, 1993.

Huang, Y et al, Mol Med, 6:647-658, 1995.

Hudziak, R. M et al., Antisense Nucleic Acid Drug Dev., 6:267-272, 1996.

Hudziak, R. M et al., Antisense Nucleic Acid Drug Dev., 10:163-176,2000.

Jones D P and Chesney R W., Curr Opin Pediatr. 7(2):208-13,1995.

Kipshidze, N et al., XXI Congress of the European Society of Cardiology, pp 463-467. Bologna (Italy): Monduzzi Editore S.p.A, 1999.

Lappalainen, et al., Biochim Biophys Acta. 1196(2):201-8,1994.

Leonetti, C et al., J Natl Cancer Inst., 88(7):419-29, 1996.

Li et al., J Surg Res., 59(4):485-92 1995).

Loke, S. L., et al., Proc Natl Acad Sci U S A. 86(10):3474-8, 1989.

Lou X et al., J Biomater Appl 15(4):307-20, 2001.

Mayo J G, Cancer Chemother. Rept. 3:325-330,1972.

McGuffie E et al., Cancer Res. 60(14):3790-9, 2000.

Monteith D K et al., Toxicol Pathol 27(3):307-17, 1999.

Nagase et al., Cancer Treatment Reports 71:825-829, 1997.

Nicolaou K C et al., Nature 367(6464):630-4, 1994.

Onoda J M et al., Cancer Lett 40(1):39-47, 1988.

Pendergast G C, Ed., Oncogene Reviews, Myc and Myb. 18(19):1, 1999.

Peters G J et al., Pharmacol Ther 87(2-3):227-53, 2000.

Popescu R A et al., Eur J Cancer, 34(8):1268-73. 1998.

Ricker J L et al., Kidney International. 2001 (Submitted).

Skorski, T et al., Proc Natl Acad Sci U S A., 94(8):3966-71, 1997.

Skorski, T et al., Antisense Nucleic Acid Drug Dev., 7:187-195, 1997.

Smith, J B and Wickstrom, E J, Natl. Cancer Inst., 15:1146-1154,1998.

Smith J B and Wickstrom E, Methods Enzymol., 314:537-80, 2000.

Spitzer, F. and Eckstein F., Nucleic Acid Res. 16: 11691-11704, 1988.

Summerton, J and Weller D, Antisense Nucleic Acid Drug Dev., 7:187-195, 1997

Summerton, J. and Weller, D. Nucleos. Nucleot. 16:889-898,1997.

Theon et al. JAVMA 202:261-267, 1993.

Toulme, J. J. et al., Biochimie 78(7):663-73, 1996.

Williams A S, Br J Rheumatol. 35(8):719-24,1996.

Wu W J and Wu T L, J Microencapsul. 16(5):63946, 1999.

Nakamura N et al., J Biochem (Tokyo) 129(5):755-9,2001.

Yakubov, L. A., et al., Proc Natl Acad Sci U S A. 86(17):6454-8, 1989.

Yuen A R and Sikic B I, Frontiers in Bioscience d588-593, 2000.

### BACKGROUND OF THE INVENTION

The development of cancer is a complex, multi-step process that has been linked to an alteration in the expression level of cellular proto-oncogenes including the proto-oncogene c-myc (Denhardt, D T, 1999; Pendergast G C, 1999). c-myc regulates cell growth, differentiation, and apoptosis, and its aberrant expression has been associated with a number of human cancers including lung cancer, colorectal cancer, breast cancer, bladder cancer, leukemia, lung cancer, etc (Dang et al., 1999). Reports implicating upregulated or aberrant expression of the basic-helix-loop-helix nuclear c-myc in numerous cancers has led to pre-clinical and clinical studies evaluating the effects c-myc inhibition using a number of approaches.

It has been demonstrated that antisense oligonucleotides and antibodies can specifically interfere with synthesis of a target protein of interest. Due to their hydrophobicity, antisense oligonucleotides interact well with phospholipid membranes (Akhtar et al., 1991), and it has been suggested that following the interaction with the cellular plasma membrane, oligonucleotides are actively transported into living cells (Loke et al., 1989; Yakubov et al., 1989; Anderson et al., 1999).

Studies have been undertaken to test antisense compounds which have a phosphorothioate backbone and are directed against seven cancer related genes including p53, bcl-2, c-raf, H-ras, protein kinase C-alpha, and protein kinase A. Side effects including transient thrombocytopenia, fatigue and fever have been observed and are attributed to the phosphorothioate backbone. In addition, inhibition of target gene expression was determined to be “modest at most”, and definitive clinical activity has not been observed (Yuen et al., 2000).

Further studies employing phosphorothioate (PSOs) and N3′-P-5′ phosphoramidate antisense oligonucleotides targeted to the c-myc translation start site have been reported to inhibit growth of various tumor cell types (Leonetti et al., 1996; Smith et al., 1998; and Skorski et al., 1997). These studies suggest that c-myc inhibitors could be clinically useful in treating proliferative diseases such as cancer and restenosis.

Phosphorodiamidate morpholino oligomers (PMOs) represent a novel antisense structural type wherein the phosphodiester linkage is replaced by an uncharged phosphoramidate linkage and the deoxyribose sugar is replaced by a morpholine ring (Summerton et al., 1997). PMOs have been demonstrated to be resistant to a variety of nucleases and proteases (Hudziak et al., 1996), bind with higher affinity to RNA than congenic phosphodiester DNA (Summerton et al., 1997), and act as steric inhibitors of translation initiation (Ghosh et al., 1999).

The c-myc antisense oligomer has been shown to inhibit normal pre-mRNA splicing and to produce aberrantly spliced mRNA (Hudziak et al., 2000). A PMO antisense to c-myc has been demonstrated to be a sequence specific inhibitor of c-myc translation in cancer cells, causing a decrease in c-myc protein expression and arrest of the cell cycle in Gₒ/G₁ and has been proposed for use in cancer therapy (Hudziak et al., 2000).

Despite advances in cancer treatment strategies, lack of efficacy and/or significant side effects due to the toxicity of currently used chemotherapeutic agents remains a problem. Drug toxicity can be severe enough to result in life-threatening situations, which require administration of drugs to counteract side effects, and may result in the reduction and/or discontinuation of the chemotherapeutic agent, which may impact negatively on the patient's treatment and/or the quality of life.

Gene therapy strategies have been attempted and are the subject of ongoing clinical trials. However, consistent with traditional chemotherapy, the lack of specificity of delivery systems and toxic side effects due to those delivery systems must be overcome in order for such strategies to have clinical relevance.

Accordingly, there remains a need for improved cancer treatment regimens which address the deficiencies in current therapeutic approaches. The present invention addresses this need.

### SUMMARY OF THE INVENTION

Therefore, an aspect of the present invention is to provide an improved method for the treatment of cancer susceptible to treatment by chemotherapy, where the improvement relates to a treatment regimen that includes administering an oligomer antisense to c-myc and a chemotherapeutic agent to a cancer patient, wherein the oligomer antisense to c-myc and the chemotherapeutic agent are to be administered sequentially and at least one day apart.

Another aspect of the invention is to provide the use of an oligomer antisense to c-myc and a chemotherapeutic agent in the preparation of a pharmaceutical composition for the treatment of cancer susceptible to chemotherapy, wherein the oligomer antisense to c-myc and chemotherapeutic agent are to be administered sequentially and at least one day apart.

A related aspect of the present invention is the provision of an oligomer composition for the treatment of cancer in a patient currently being treated by chemotherapy, comprising an oligomer antisense to c-myc, wherein the composition is administered prior to or following administration of a chemotherapeutic agent.

Yet another aspect of the invention is to provide kits for the treatment of cancer susceptible to treatment by chemotherapy. Such kits include a first composition comprising an oligomer antisense to c-myc and a second composition comprising a chemotherapeutic agent, wherein the first composition and second composition are to be administered sequentially and at least one day apart.

Preferably, the antisense oligomers have a length of about 12 to 25 bases and are characterized by:

(a) a backbone which is substantially uncharged;

(b) the ability to hybridize with the complementary sequence of a target RNA with high affinity at a Tm greater than 50° C.;

(c) nuclease resistance; and

(d) the capability for active or facilitated transport into cells.

Preferably, antisense oligomers are targeted to a sequence spanning the mRNA translational start codon for c-myc or a splice acceptor region of c-myc mRNA. Examples of preferred c-myc antisense oligomer sequences for use in practicing the invention include oligomers containing the sequence presented as SEQ ID NO:1, SEQ ID NO:8, SEQ ID NO:9, SEQ ID NO:10, and SEQ ID NO:11.

These and other objects and features of the invention will become more fully apparent when the following detailed description is read in conjunction with the accompanying figures and examples.

### BRIEF DESCRIPTION OF THE FIGURES

FIG. 1 shows several preferred morpholino-type subunits having 5-atom (A), six-atom (B) and seven-atom (C-D) linking groups suitable for forming polymers;

FIGS. 2A-D show the repeating subunit segment of exemplary morpholino oligonucleotides, designated A through D, constructed using subunits A-D, respectively, of FIG. 1.

FIGS. 3A-3G show examples of uncharged linkage types in oligonucleotide analogs.

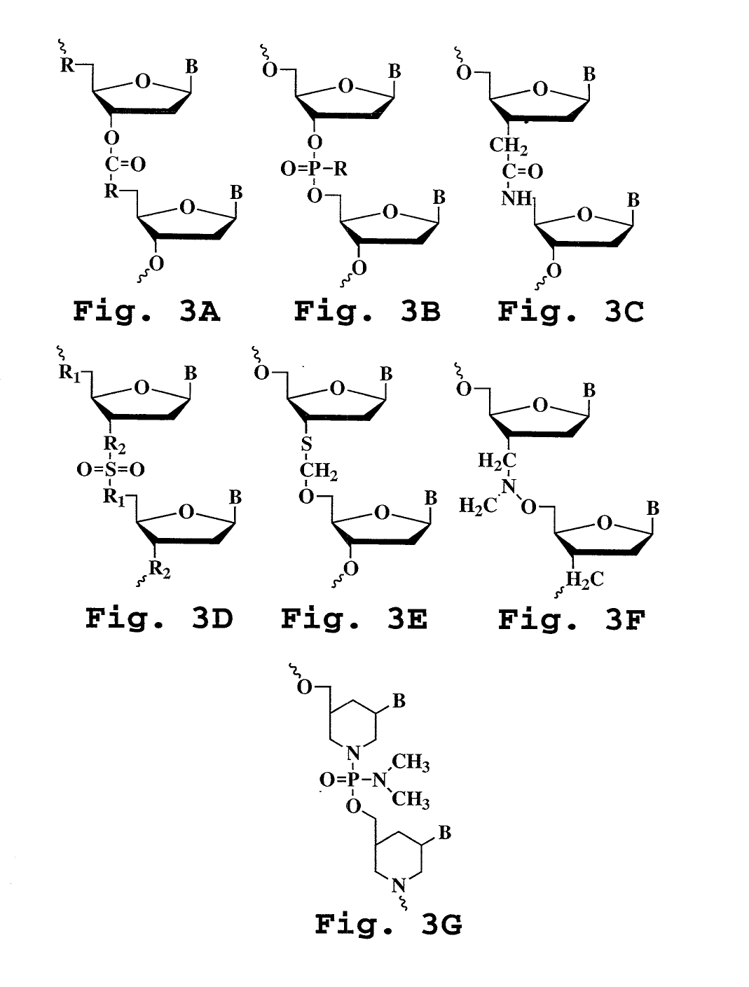

FIGS. 4A-C depict reverse HPLC chromatograms representative of tumor tissue from LL tumor bearing mice treated with a single i.p. injection of saline or AVI-4126. The Figures provides reference control chromatograms (FIG. 4A), chromatograms representative of tumor lysates from mice treated with saline (FIG. 4B) or 300 μg AVI-4126 (FIG. 4C).

FIGS. 5A and B depict the results of representative immunoblot analyses of c-myc and, β-actin protein in lysates from large, established LL tumor bearing mice given a single injection of saline (lanes 1 and 2); 100 μg c-myc scrambled control oligomer antisense oligomer (SEQ ID NO:2; lanes 3 and 4); or 100 μg AVI-4126 antisense oligomer (SEQ ID NO:1; lanes 5 and 6); where lane 7 is a positive control for c-myc.

FIGS. 6A-C provide an image of a Western blot of representative tumor lysates from saline (lanes 2-5) and AVI-4126 (lanes 6-9) treated mice. Lane 1 is a c-myc positive control, where panel A was probed with an N-terminal c-myc antibody, panel B was probed with a C-terminal c-myc antibody and panel C was probed with a β-actin antibody and serves as a loading control.

FIGS. 7A and B provide an image of a Western blot of representative tumor lysates from saline (lanes 1-2), cisplatin (lanes 3-4) and cisplatin+AVI-4126 (lanes 5-6) treated groups. FIG. 4A illustrates the results when the blot was probed with an N-terminal c-myc antibody and FIG. 4B illustrates the results when the blot was probed with a β-actin antibody as a loading control.

FIGS. 8A-D illustrate the effects of AVI-4126 in combination chemotherapy treatment as described in Table 1, where AVI4126 is administered in an alternating treatment regimen with cisplatin (FIG. 8A), Taxol (FIG. 8B), etoposide (FIG. 8C) and 5-FU (FIG. 8D).

### DETAILED DESCRIPTION OF THE INVENTION

I. Definitions

The terms below, as used herein, have the following meanings, unless indicated otherwise:

As used herein, the terms “compound”, “agent”, “oligomer” and “oligonucleotide” may be used interchangeably with respect to the antisense oligonucleotides of the invention. Similarly, the terms “compound” and “agent” may be used interchangeably with respect to the chemotherapeutic compounds for use in practicing the invention.

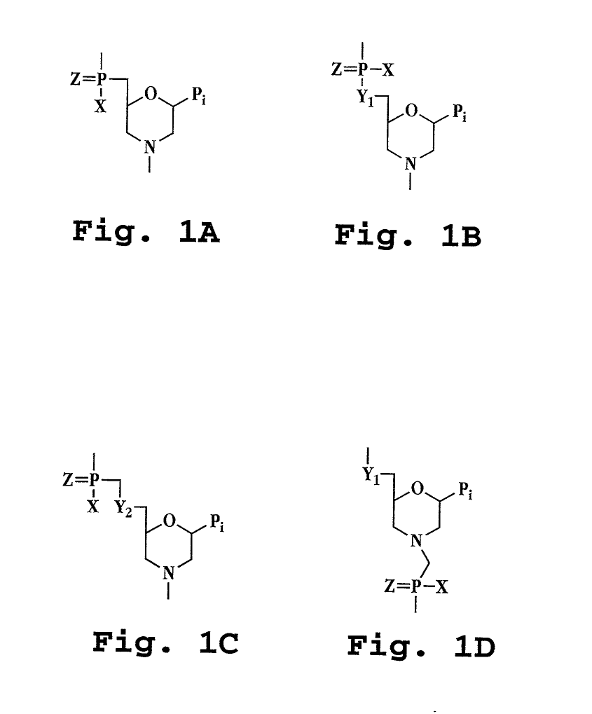

As used herein, the terms “antisense oligonucleotide” and “antisense oligomer” are used interchangeably and refer to a sequence of nucleotide bases and a subunit-to-subunit backbone that allows the antisense oligomer to hybridize to a target sequence in an RNA by Watson-Crick base pairing, to form an RNA:oligomer heteroduplex within the target sequence. The oligomer may have exact sequence complementarity to the target sequence or near complementarity. Such antisense oligomers may block or inhibit translation of the mRNA containing the target sequence, or inhibit gene transcription, may bind to double-stranded or single stranded sequences, and may be said to be “directed to” a sequence with which it hybridizes. Exemplary structures for antisense oligonucleotides for use in the invention include the β-morpholino subunit types shown in FIGS. 1A-E. It will be appreciated that a polymer may contain more than one linkage type.

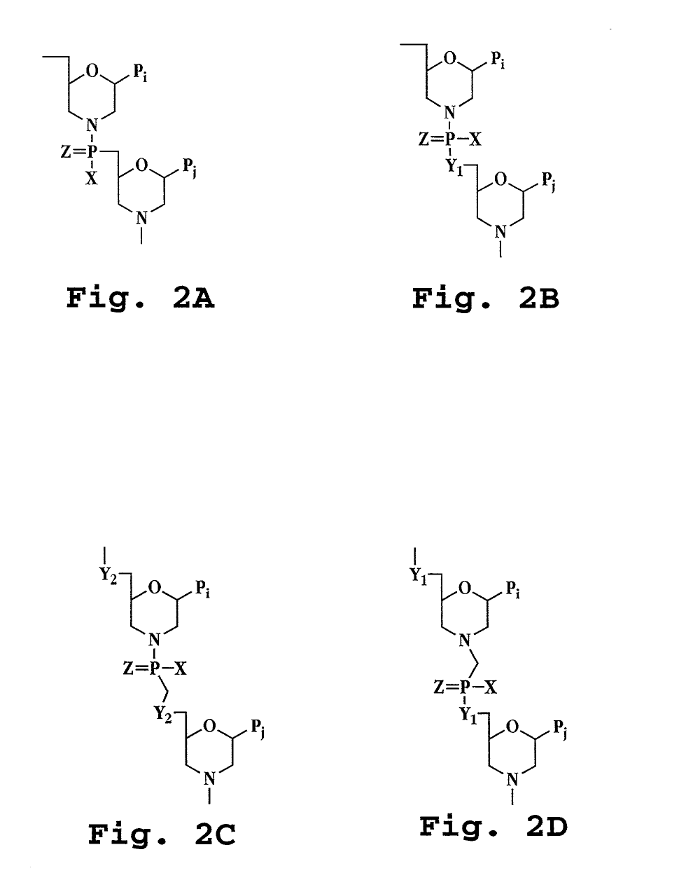

Subunit A in FIG. 1 contains a 1-atom phosphorous-containing linkage which forms the five atom repeating-unit backbone shown at A of FIG. 2, where the morpholino rings are linked by a 1-atom phosphonamide linkage.

Subunit B in FIG. 1 is designed for 6-atom repeating-unit backbones, as shown at B, in FIG. 2. In structure B of FIG. 1, the atom Y linking the 5′ morpholino carbon to the phosphorous group may be sulfur, nitrogen, carbon or, preferably, oxygen. The X moiety pendant from the phosphorous may be any of the following: fluorine; an alkyl or substituted alkyl; an alkoxy or substituted alkoxy; a thioalkoxy or substituted thioalkoxy; or, an unsubstituted, monosubstituted, or disubstituted nitrogen, including cyclic structures.

Subunits C-E in FIG. 1 are designed for 7-atom unit-length backbones as shown for C through E in FIG. 2. In Structure C of FIG. 1, the X moiety is as in Structure B of FIG. 1 and the moiety Y may be a methylene, sulfur, or preferably oxygen. In Structure D of FIG. 1 the X and Y moieties are as in Structure B of FIG. 1. In Structure E of FIG. 1, X is as in Structure B of FIG. 1 and Y is O, S, or NR. In all subunits depicted in FIGS. 1A-E, Z is O or S, and Pᵢ or Pⱼ is adenine, cytosine, guanine or uracil.

As used herein, a “morpholino oligomer” refers to a polymeric molecule having a backbone which supports bases capable of hydrogen bonding to typical polynucleotides, wherein the polymer lacks a pentose sugar backbone moiety, and more specifically a ribose backbone linked by phosphodiester bonds which is typical of nucleotides and nucleosides, but instead contains a ring nitrogen with coupling through the ring nitrogen. A preferred “morpholino” oligonucleotide is composed of morpholino subunit structures of the form shown in FIG. 2B, where (i) the structures are linked together by phosphorous-containing linkages, one to three atoms long, joining the morpholino nitrogen of one subunit to the 5′ exocyclic carbon of an adjacent subunit, and (ii) Pᵢ and Pⱼ are purine or pyrimidine base-pairing moieties effective to bind, by base-specific hydrogen bonding, to a base in a polynucleotide.

This preferred aspect of the invention is illustrated in FIG. 2B, which shows two such subunits joined by a phosphorodiamidate linkage. Morpholino oligonucleotides (including antisense oligomers) are detailed, for example, in co-owned U.S. Pat. Nos. 5,698,685, 5,217,866, 5,142,047, 5,034,506, 5,166,315, 5,185,444, 5,521,063, and 5,506,337, all of which are expressly incorporated by reference herein.

As used herein, a “nuclease-resistant” oligomeric molecule (oligomer) is one whose backbone is not susceptible to nuclease cleavage of a phosphodiester bond. Exemplary nuclease resistant antisense oligomers are oligonucleotide analogs, such as phosphorothioate and phosphate-amine DNA (pnDNA), both of which have a charged backbone, and methyl-phosphonate, morpholino, and peptide nucleic acid (PNA) oligonucleotides, all of which may have uncharged backbones.

As used herein, an oligonucleotide or antisense oligomer “specifically hybridizes” to a target polynucleotide if the oligomer hybridizes to the target under physiological conditions, with a Tm substantially greater than 37° C., preferably at least 50° C., and typically 60° C.-80° C. or higher. Such hybridization preferably corresponds to stringent hybridization conditions, selected to be about 10° C., and preferably about 5° C. lower than the thermal melting point (T[m]) for the specific sequence at a defined ionic strength and pH. At a given ionic strength and pH, the T_{[m]} is the temperature at which 50% of a target sequence hybridizes to a complementary polynucleotide.

Polynucleotides are described as “complementary” to one another when hybridization occurs in an antiparallel configuration between two single-stranded polynucleotides. A double-stranded polynucleotide can be “complementary” to another polynucleotide, if hybridization can occur between one of the strands of the first polynucleotide and the second. Complementarity (the degree that one polynucleotide is complementary with another) is quantifiable in terms of the proportion of bases in opposing strands that are expected to form hydrogen bonds with each other, according to generally accepted base-pairing rules.

As used herein the term “analog” with reference to an oligomer means a substance possessing both structural and chemical properties similar to those of a reference oligomer.

As used herein, a first sequence is an “antisense sequence” with respect to a second sequence if a polynucleotide whose sequence is the first sequence specifically binds to, or specifically hybridizes with, the second polynucleotide sequence under physiological conditions.

As used herein, a “base-specific intracellular binding event involving a target RNA” refers to the sequence specific binding of an oligomer to a target RNA sequence inside a cell. For example, a single-stranded polynucleotide can specifically bind to a single-stranded polynucleotide that is complementary in sequence.

As used herein, “nuclease-resistant heteroduplex” refers to a heteroduplex formed by the binding of an antisense oligomer to its complementary target, which is resistant to in vivo degradation by ubiquitous intracellular and extracellular nucleases.

As used herein, “c-myc”, refers to an oncogene or gene that gives directs cells toward the development and growth of cancer or a tumor. “c-myc” has been associated with gene amplification in various types of cancer, as further detailed below.

As used herein, the term “c-myc antisense oligomer” refers to a nuclease-resistant antisense oligomer having high affinity (ie, which “specifically hybridizes”) to a complementary or near-complementary c-myc nucleic acid sequence.

As used herein, the term “modulating expression” relative to an oligonucleotide refers to the ability of an antisense oligonucleotide (oligomer) to either enhance or reduce the expression of a given protein by interfering with the expression, or translation of RNA. In the case of enhanced protein expression, the antisense oligomer may block expression of a suppressor gene, e.g., a tumor suppressor gene. In the case of reduced protein expression, the antisense oligomer may directly block expression of a given gene, or contribute to the accelerated breakdown of the RNA transcribed from that gene.

As used herein, the terms “tumor” and “cancer” refer to a cell that exhibits a loss of growth control and forms unusually large clones of cells. Tumor or cancer cells generally have lost contact inhibition and may be invasive and/or have the ability to metastasize.

As used herein, “effective amount” relative to an antisense oligomer refers to the amount of antisense oligomer administered to a mammalian subject, either as a single dose or as part of a series of doses and which is effective to inhibit expression of a selected target nucleic acid sequence.

As used herein “treatment” of an individual or a cell is any type of intervention used in an attempt to alter the natural course of the individual or cell. Treatment includes, but is not limited to, administration of e.g., a pharmaceutical composition, and may be performed either prophylactically, or subsequent to the initiation of a pathologic event or contact with an etiologic agent.

II. Antisense Oligonucleotides for use in Practicing the Invention

A. Types of Antisense Oligonucleotides

Antisense oligonucleotides of 15-20 bases are usually long enough to have one complementary sequence in the mammalian genome. In addition, antisense compounds having a length of at least 17 nucleotides have been shown to hybridize well with a complementary target mRNA sequence (Cohen et al., 1991).

Two general mechanisms have been proposed to account for inhibition of expression by antisense oligonucleotides. (See e.g., Agrawal et al., 1990; Bonham et al., 1995; and Boudvillain et al., 1997.) In the first, a heteroduplex formed between the oligonucleotide and mRNA is a substrate for RNase H, leading to cleavage of the mRNA. Oligonucleotides belonging, or proposed to belong, to this class include phosphorothioates, phosphotriesters, and phosphodiesters (i.e., unmodified “natural” oligonucleotides). Such compounds generally show high activity, and phosphorothioates are currently the most widely employed oligonucleotides in antisense applications. However, these compounds tend to produce unwanted side effects due to non-specific binding to cellular proteins (Gee et al., 1998), as well as inappropriate RNase cleavage of non-target RNA heteroduplexes (Giles et al., 1993).

A second class of oligonucleotide analogs, termed “steric blockers” or, alternatively, “RNase H inactive” or “RNase H resistant”, have not been observed to act as a substrate for RNase H, and are believed to act by sterically blocking target RNA formation, nucleocytoplasmic transport or translation. This class includes methylphosphonates (Toulme et al., 1996), morpholino oligonucleotides, peptide nucleic acids (PNA's), 2′-O-allyl or 2′-O-alkyl modified oligonucleotides (Bonham, 1995), and N3′→P5′ phosphoramidates (Gee, 1998).

Naturally occurring oligonucleotides have a phosphodiester backbone which is sensitive to degradation by nucleases; however, certain modifications of the backbone increase the resistance of native oligonucleotides to such degradation. (See, e.g., Spitzer et al., 1988.)

B. Preferred Antisense Oligonucleotides

In addition to a base sequence complementary to a region of a selected nucleic acid target sequence, preferred antisense oligonucleotides exhibit highly specific binding to the complementary target sequence and efficacy in blocking expression of the target nucleic acid in cell and cell-free systems.

Antisense oligomers for use in the methods of the invention preferably, have one or more properties including: (1) a backbone that is substantially uncharged (e.g., Uhlmann, et al., 1990), (2) the ability to hybridize with the complementary sequence of a target RNA with high affinity, that is a Tm substantially greater than 37° C., preferably at least 50° C., and typically 60° C.-80° C. or higher, (3) a subunit length of at least 8 bases, generally about 8-40 bases, preferably 12-25 bases, (4) nuclease resistance (Hudziak, et al., 1996) and (5) capability for active or facilitated transport as evidenced by (i) competitive binding with a phosphorothioate antisense oligomer, and/or (ii) the ability to transport a detectable reporter into cells.

Morpholino oligonucleotides, particularly phosphoramidate- or phosphorodiamidate-linked morpholino oligonucleotides have been shown to have high binding affinities for complementary or near-complementary nucleic acids. Morpholino oligomers also exhibit little or no non-specific antisense activity, afford good water solubility, are immune to nucleases, and are designed to have low production costs (Summerton et al., 1997).

The synthesis, structures, and binding characteristics of morpholino oligomers are detailed in U.S. Pat. Nos. 5,698,685; 5,217,866; 5,142,047; 5,034,506; 5,166,315; 5,521,063; and 5,506,337, each of which are expressly incorporated by reference herein.

The preferred antisense oligomers of the present invention are composed of morpholino subunits of the form shown in the above cited patents, where (i) the morpholino groups are linked together by uncharged linkages, one to three atoms long, joining the morpholino nitrogen of one subunit to the 5′ exocyclic carbon of an adjacent subunit, and (ii) the base attached to the morpholino group is a purine or pyrimidine base-pairing moiety effective to bind, by base-specific hydrogen bonding, to a base in a polynucleotide. The purine or pyrimidine base-pairing moiety is typically adenine, cytosine, guanine, uracil or thymine. Preparation of such oligomers is described in detail in U.S. Pat. No. 5,185,444 (Summerton et al., 1993), which is hereby incorporated by reference in its entirety. As shown in the reference, several types of nonionic linkages may be used to construct a morpholino backbone.

Exemplary backbone structures for antisense oligonucleotides of the invention include the β-morpholino subunit types shown in FIGS. 1A-E. It will be appreciated that a polynucleotide may contain more than one linkage type.

Subunit A in FIG. 1 contains a 1-atom phosphorous-containing linkage which forms the five atom repeating-unit backbone shown at A in FIG. 2, where the morpholino rings are linked by a 1-atom phosphoamide linkage.

Subunit B in FIG. 1 is designed for 6-atom repeating-unit backbones, as shown at B in FIG. 2. In structure B, the atom Y linking the 5′ morpholino carbon to the phosphorous group may be sulfur, nitrogen, carbon or, preferably, oxygen. The X moiety pendant from the phosphorous may be any of the following: fluorine; an alkyl or substituted alkyl; an alkoxy or substituted alkoxy; a thioalkoxy or substituted thioalkoxy; or, an unsubstituted, monosubstituted, or disubstituted nitrogen, including cyclic structures. In a preferred embodiment, the X moiety pendant from the phosphorous is a dimethyl amino group [N(CH₃)₂].

Subunits C-E in FIG. 1 are designed for 7-atom unit-length backbones as shown for C through E in FIG. 2. In Structure C of FIG. 1, the X moiety is as in Structure B of FIG. 1 and the moiety Y may be a methylene, sulfur, or preferably oxygen. In Structure D of FIG. 1 the X and Y moieties are as in Structure B of FIG. 1. In Structure E of FIG. 1, X is as in Structure B and Y is O, S, or NR. In all subunits depicted in FIGS. 1A-E, Z is O or S, and Pᵢ or Pⱼ is adenine, cytosine, guanine or uracil.

A preferred “morpholino” oligonucleotide is composed of morpholino subunit structures of the form shown in FIG. 2B, where (i) the structures are linked together by phosphorous-containing linkages, one to three atoms long, joining the morpholino nitrogen of one subunit to the 5′ exocyclic carbon of an adjacent subunit and (ii) Pᵢ and Pⱼ are purine or pyrimidine base-pairing moieties effective to bind, by base-specific hydrogen bonding, to a base in a polynucleotide.

C. Preferred Antisense Targets

In practicing the invention, mRNA transcribed from the relevant region of a gene of interest is generally targeted by antisense oligonucleotides; however, single-stranded RNA, double-stranded RNA, single-stranded DNA or double-stranded DNA may be targeted. For example, double-stranded DNA may be targeted using a non-ionic probe designed for sequence-specific binding to major-groove sites in duplex DNA. Exemplary probes are described in U.S. Pat. No. 5,166,315 (Summerton and Weller, 1992), which is hereby incorporated by reference. Such probes are generally referred to herein as antisense oligomers, referring to their ability to block expression of target nucleic acids.

In the methods of the invention, the antisense oligomer is designed to hybridize to a region of the c-myc nucleic acid sequence, under physiological conditions with a Tm substantially greater than 37° C., e.g., at least 50° C. and preferably 60° C.-80° C. The oligomer is designed to have high-binding affinity to the nucleic acid and may be 100% complementary to the c-myc target sequence or may include mismatches, e.g., to accommodate allelic variants, as long as the heteroduplex formed between the oligomer and c-myc target sequence is sufficiently stable to withstand the action of cellular nucleases and other modes of degradation during its transit from cell to body fluid. Mismatches, if present, are less destabilizing toward the end regions of the hybrid duplex than in the middle. The number of mismatches allowed will depend on the length of the oligomer, the percentage of G:C base pair in the duplex and the position of the mismatch(es) in the duplex, according to well understood principles of duplex stability.

Although such an antisense oligomer is not necessarily 100% complementary to the c-myc target sequence, it is effective to stably and specifically bind to the target sequence such that expression of c-myc is modulated. The appropriate length of the oligomer to allow stable, effective binding combined with good specificity is about 8-40 nucleotide base units, and preferably about 12-25 nucleotides. Oligomer bases that allow degenerate base pairing with target bases are also contemplated, assuming base-pair specificity with the target is maintained.

In one preferred approach, the target for modulation of gene expression using the antisense methods of the present invention comprises a sequence spanning the mRNA translational start codon for c-myc. In an alternative preferred approach, a splice acceptor region of c-myc mRNA is targeted. It will understood that other regions of c-myc mRNA may be targeted, including one or more of, an initiator or promoter site, an intron or exon junction site, a 3′-untranslated region, and a 5′-untranslated region. It will be further understood that both spliced and unspliced RNA may serve as the template for design of antisense oligomers for use in the methods of the invention. (See, e.g., Hudziak et al., 2000, expressly incorporated by reference herein.)

Hudziak et al., 2000 describe a number of oligomers antisense to c-myc mRNA that were shown to have antiproliferative effects on transformed human and rat fibroblast cells (NRK and WI-38, respectively). Exemplary antisense oligomers are provided in Table 1, below.

TABLE 1

Exemplary Antisense Oligomers

| | | | |
|---|---|---|---|
| Oligomer | | Length | Spe- |
| # | Sequence¹ | (bases) | cies |

| | | | |
|---|---|---|---|
| 92 | (SEQ ID NO:5) | 21 | R,H |
| | GMT MMM TMT GTM TMT MGM TGG | | |
| 93 | (SEQ ID NO:6) | 20 | R,H |
| | MMG MMM GMT MGM TMM MTM TG | | |
| 25 | (SEQ ID NO:7) | 30 | R |
| | GGC AUC GUC GUG ACU GUC GGG | | |
| | UUU UCC ACC | | |
| 21 | (SEQ ID NO:8) | 30 | R |
| | GGG GCA UCG UCG UGA CUG UCU | | |
| | GUU GGA GGG | | |
| 108 | (SEQ ID NO:9) | 22 | R |
| | CGU CGU GAC UGU CUG UUG GAG | | |
| 111 | (SEQ ID NO:10) | 22 | R |
| | CGT CGT GAC TGT CTG TTG GAG G | | |
| 37 | (SEQ ID NO:11) | 28 | H |
| | GGC AUC GUC GCG GGA GGC UGC | | |
| | UGG AGC G | | |
| 26 | (SEQ ID NO:12) | 28 | R |
| | CCG CGA CAU AGG ACG GAG AGC | | |
| | AGA GCC C | | |
| 126 | (SEQ ID NO:1) | 20 | R,H |
| | ACG TTG AGG GGC ATC GTC GC | | |
| 174 | (SEQ ID NO:13) | 12 | R,H |
| | TTG AGG GGC ATC | | |

¹ all sequences are shown in the 5′ to 3′ direction; M refers to 5-methyl cytosine; T refers to thymine, R means the sequence is complementary to the rat c-myc sequence and H means the sequence is complementary to the human sequence

In exemplary embodiments of the invention, the antisense oligomer is a PMO containing the sequence presented as SEQ ID NO:1, SEQ ID NO:8, SEQ ID NO:9; SEQ ID NO:10, or SEQ ID NO:11.

III. c-myc

c-myc is a proto-oncogene that regulates cell growth, differentiation, and apoptosis, and its aberrant expression is frequently observed in human cancer. Aberrant, constitutive or overexpression of c-myc has been associated with a number of human cancers including lung cancer, colorectal cancer, breast cancer, bladder cancer, leukemia, lung cancer, etc. (See, e.g., Bieche et al., 1999.)

Proto-oncogenes are activated to oncogenes by a variety of mechanisms which include: (1) promoter insertion, (2) enhancer insertion, (3) chromosomal translocation, (4) gene amplification and (5) point mutation. As used herein, “activation” relative to a proto-oncogene means transcription of the gene is increased, e.g., from no expression to low level expression. Mechanisms (1)-(4) result in an increase in the expression level of an oncogene, while (5) results in expression of an altered gene product. Evidence suggests that some form of oncogene expression together with inactivation of tumor suppressor genes is required for the development of cancer.

The myc proto-oncogenes have been described as transcription factors that directly regulate the expression of other genes, examples of which include ECA39, p53, ornithine decarboxylase (ODC), alpha-prothymosin and Cdc25A (Ben-Yosef et al., 1998).

In chickens, following infection of chicken B-cells with certain avian leukemia viruses, a provirus becomes integrated near the myc gene, which is activated by a viral long terminal repeat (LTR) that acts either as a promoter or an enhancer, resulting in expression of myc and formation of a B-cell. Similarly, in Burkift's lymphoma, an enhancer sequence is translocated resulting in expression of myc. (See, e.g., Gauwerky et al., 1993).

c-myc is expressed in normal hematopoietic stem cells and has been shown to promote the differentiation of human epidermal stem cells (Gandarillas et al., 1997). It has been observed that when quiescent cells re-enter the cell cycle c-myc expression is up-regulated, and that ectopic expression of c-myc prevents cell cycle arrest in response to growth-inhibitory signals, differentiation stimuli, or mitogen withdrawal. (See, e.g., Amati et al., 1998.) Further, the expression of an apoptosis inhibitor, bcl-2 has been inversely correlated with expression of c-myc in colorectal cancer cells. (See, e.g., Popescu R A et al., 1998.)

Following c-myc antisense phosphorothioate oligomer treatment of c-myc over-expressing leukemia and colon cancer cell lines, inhibition of cellular proliferation was observed together with detection of a 20- to 100-fold decrease in c-myc mRNA in the colon cancer cell line and the leukemic cell line, respectively, using a competitive reverse transcription-polymerase chain reaction (Li et al., 1995. See, also, McGuffie et al., 2000; Skorski et al., 1997 and Huang et al., 1995). In addition, oligodeoxynucleotides antisense to c-myc mRNA protein binding site targets were demonstrated to inhibit RNA binding by 75% in a sequence-specific manner. K562 cells treated with such a c-myc antisense oligonucleotide showed a concentration-dependent decrease in both c-myc mRNA and protein levels. In contrast, a c-myc antisense oligonucleotide targeting the translation initiation codon was shown to reduce c-myc protein but increased mRNA levels (Coulis et al., 2000).

Furthermore, studies on the renal effects of phosphorothioate oligodeoxynucleotides in monkeys indicated nonspecific and evidence of toxicity. The compounds were shown to accumulate in the kidney and induce proximal tubular degeneration at high doses (Monteith et al., 1999). This may be due to the charged nature of phosphorothioate oligonucleotides resulting in co-precipitation with the chemotherapeutic agent and accumulation of the and precipitate in the kidney. In contrast, unlike the charged phosphorothioate oligonucleotides, the PMOs of the invention are substantially uncharged and therefore lack a site for interaction or co-precipitation with a chemotherapeutic agent such as cispaltin.

Surprisingly, when an antisense oligomer to c-myc was used to treat an enriched population of hematopoietic stem cells, development of the hematopoietic stem cell population was modulated, as described in co-owned U.S. application Ser. No. 09/679,475 (PCT publication number, WO 01/25405).

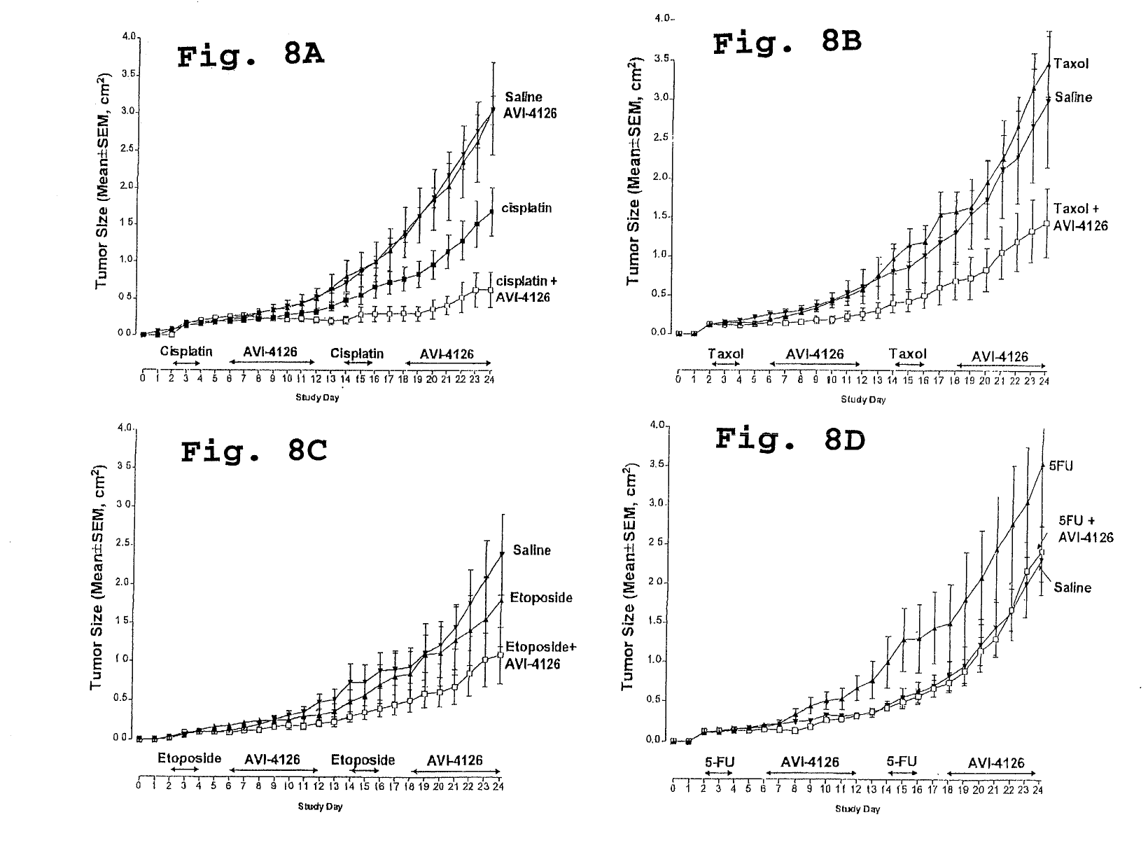

The present invention reflects the surprising discovery that when an oligomer antisense to c-myc is used in combination with several widely used chemotherapeutic agents, enhanced anti-cancer efficacy results. As can be seen from the results presented in Example 1, using a model which employs Lewis lung cell derived tumors in C57BL mice, inhibition of c-myc expression in tumors treated with an oligomer antisense to c-myc (AVI4126, SEQ ID NO:1) was found to be dependent upon the timing of administration of the antisense oligomer relative to cisplatin treatment. Further, the c-myc antisense oligomer was shown to significantly enhance the anti-tumor activity of cisplatin, etoposide and taxol but not 5-FU. (See FIGS. 8A-D.)

IV. Traditional Cancer Treatment Regimens

Current cancer therapeutic regimens suffer from a number of deficiencies the most important of which are a lack of efficacy and frequent toxic side effects. One of the major limitations to clinical use of cancer therapeutic agents is the development of resistance to the treatment. The problem of drug resistance has been observed with a number of chemotherapeutic agents, including cisplatin-type compounds used to treat solid tumors and leukemias. Such resistance is typically evidenced by recurrence of the tumor subsequent to chemotherapy. As a result, most therapeutic regimes include two or more different drugs as a method of circumventing resistance. In addition, high dose chemotherapy is typically required for effective treatment. Such high doses are associated with toxic side effects.

Chemotherapeutic agents for use in practicing the invention include any of a number of agents with established use in cancer therapy. Exemplary chemotherapeutic agents for use in the invention are antimetabolities, compounds which cause oxidative stress, and topoisomerase inhibitors. Without being bound to any one particular theory, it is believed that chemotherapeutic agents are more toxic to less differentiated cells and as such, a population of more highly differentiated cancer cells that are refractory to the chemotherapeutic agent remain after chemotherapy treatment. Such cells may be more differentiated and accordingly, more susceptible to inhibition or cell death by a c-myc antisense oligomer.

Exemplary anticancer drugs include, but are not limited to: (1) antimetabolites such as folic acid analogs and methotrexate, (MTX); pyrimidine analogs such as 5-fluorouracil, (5-FU), fluorodeoxyuridine, cytosine arabinoside and cytarabine; purine analogs such as 6-mercaptopurine, (6-MP) and 6-thioguanine, (6-TG); (2) alkylating agents such as nitrogen mustards, mechlorethamine, cyclophosphamide (CytoxanR), melphalan, and chlorambucil; (3) natural products including, but not limited to vinca alkaloids, vincristine (OncovinR), vinblastine (VelbanR), vinorelbine (NavelbineR), epipodophylotoxins, etoposide (VePesidR, VP-16) and taxol (PaclitaxeiR); (4) compounds characterized as anti-tumor antibiotics which include, but are not limited to anthracyclines, doxorubicin hydrochloride, (adriamycinR), daunorubicin, idarubicin, mitoxantrone, bleomycin, (blenoxaneR), dactinomycin (actinomycin D), mitomycin C, plycamycin and (mithramycin); and (5) miscellaneous agents including, but not limited to cisplatin, carboplatin, asparaginase, hydroxyurea, mitotane (o,p′-DDD; Lysodren), tamoxifen and prednisone.

Cisplatin (also called cis-platinum, platinol; cis-diamminedichloroplatinum; and cDDP) is representative of a broad class of water-soluble, platinum coordination compounds frequently employed in the therapy of testicular cancer, ovarian tumors, and a variety of other cancers. (See, e.g., Blumenreich et al., 1985; Forastiere et al., 2001.) Methods of employing cDDP clinically are well known in the art. For example, cDDP has been administered in a single day over a six hour period, once per month, by slow intravenous infusion. For localized lesions, cDDP can be administered by local injection. Intraperitoneal infusion can also be employed. cDDP can be administered in doses as low as 10 mg/m² per treatment if part of a multi-drug regimen, or if the patient has an adverse reaction to higher dosing. In general, a clinical dose is from about 30 to about 120 or 150 mg/m² per treatment.

Typically, platinum-containing chemotherapeutic agents are administered parenterally, for example by slow intravenous infusion, or by local injection, as discussed above. The effects of intralesional (intratumoral) and IP administration of cisplatin is described in Nagase et al., 1997 and Theon et al., 1993.

Although cisplatin is widely used, side effects reported following administration of cisplatin (CDDP or Platinol), are common and include thinned or brittle hair, loss of appetite and/or weight, diarrhea, nausea and vomiting, and numbness or tingling in the fingertips and toes. In general, the effects of cisplatin are non-specific and administration of cisplatin results in damage to all rapidly growing tissues. See, e.g., Gandara et al., 1991; Peters et al., 2000; Jones et al., 1995; and Byhardt R W, 1995).

Further, cisplatin is effective against a narrow range of tumors and the development of resistance has been reported (Onoda et al., 1988).

Taxol (Paclitaxel) is a complex diterpenoid originally isolated in small yields from the bark of various species of yew (Taxaceae). Taxol can now also be prepared by chemical synthesis. (See, e.g., Nicolaou et al., 1994.) Taxol constitutes one of the most potent drugs in cancer chemotherapy and has been approved by FDA for treatment of ovarian and breast cancer and has exhibited potential utility in the treatment of lung, skin, and head/neck cancers.

The clinical utility of taxol and related drugs has been limited by cost, limited bioavailability (due to of low aqueous solubility), and the development of multiresistant cells. Solubilizers, such as Cremophor (polyethoxylated castor oil) and alcohol have been demonstrated to improve the solubility and microencapsulated forms have been described. (See, e.g, WO 93/18751) In general, side effects reported for taxol (paclitaxel), include a reduction in white and red blood cell counts, infection, nausea and vomiting, loss of appetite, change in taste, hair loss, joint and muscle pain, numbness in the extremities and diarrhea.

Etoposide (etoposide (VP-16, VePesid Oral) is currently used in therapy for a variety of cancers, including testicular cancer, lung cancer, lymphoma, neuroblastoma, non-Hodgkin's lymphoma, Kaposi's Sarcoma, Wilms' Tumor, various types of leukemia, and others.

Etoposide is generally administered orally or intravenously. Side effects associated with administration of Etoposide Oral (VP-16, VePesid Oral) include nausea and vomiting, loss of appetite, diarrhea, stomach pain, fatigue and hair loss. The primary dose-limiting side effect of etoposide and related compounds is neutropenia, which is often severe, particularly among patients under treatment with additional chemotherapeutic agents or radiation.

5-FU, (Fluorouracil, Tradenames: 5-FU, Adrucil) has been used for chemotherapy for a variety of cancers, including colon cancer, rectal cancer, breast cancer, stomach cancer, pancreatic cancer, ovarian cancer, cervical cancer, bladder cancer vaginal warts, and actinic keratosis (a type of precancerous skin lesion). 5-FU is typically administered by intravenous (IV) injection, IV infusion (drip), orally, or as a cream applied directly to the skin. 5-FU has been associated with widely documented side effects including hair loss, headache, weakness, achiness, sensitivity of skin to sunlight, blistering skin or acne, loss of appetite and/or weight and tingling in the hands or feet.

Given the extensive side effects and lack of long term efficacy of current chemotherapeutic treatment regimens, new or improved cancer treatment regimens that reduce or eliminate such side effects and/or exhibit enhanced therapeutic efficacy would be of significant value to the medical community.

V. Treatment of Cancer Using the Methods of the Invention

The invention provides methods for treatment of cancer with an antisense oligonucleotide directed against a nucleic acid sequence encoding c-myc, together with a traditional cancer treatment, i.e., chemotherapy and/or radiation therapy.

The invention is based on the discovery that a stable, substantially uncharged antisense oligonucleotide, characterized by high Tm, capable of active or facilitated transport into cells, and capable of binding with high affinity to a complementary or near-complementary c-myc nucleic acid sequence, can be administered to a cancer patient, inhibit expression of c-myc by a cell, and when administered in combination with a traditional chemotherapeutic agent results in modulation of tumor growth.

A. Treatment of Cancer

In vivo administration of a c-myc antisense oligomer to a subject together with a traditional cancer treatment, using the methods described herein can result in an improved therapeutic outcome for the patient, dependent upon a number of factors including (1) the duration, dose and frequency of c-myc antisense oligomer administration, (2) the duration, dose, frequency and compound used for chemotherapy, (3) the duration and timing of c-myc antisense oligomer administration relative to administration of the chemotherapeutic agent, and (4) the general condition of the subject.

In general, an improved therapeutic outcome relative to a cancer patient refers to a slowing or diminution of the growth of cancer cells or a solid tumor, or a reduction in the total number of cancer cells or total tumor burden.

In preferred applications of the method, the subject is a human subject. The subject may also be a cancer patient, in particular a patient diagnosed as having a form of leukemia, lymphoma, neuroblastoma, breast cancer, colon cancer, lung cancer, or any type of cancer where the patient is being treated or has been treated with chemotherapy or radiation therapy. The method is also applicable to treatment of acute or chronic myelogenous leukemia, cholangiocarcinoma, melanoma, multiple myeloma, osteosarcoma, gastric sarcoma, glioma, bladder, cervical, colorectal, ovarian, pancreatic, prostrate, and stomach cancer.

Chemotherapy and/or radiation therapy alone or in combination with stem cell transplantation are standard treatment regimens for a number of malignancies, including acute lymphocytic leukemia, chronic myelogenous leukemia, neuroblastoma, lymphoma, breast cancer, colon cancer, lung cancer, ovarian cancer, thymomas, germ cell tumors, multiple myeloma, melanoma, testicular cancer, lung cancer, and brain cancer.

Many cancer treatment regimens result in immunosuppression of the patient, leaving the patient with anemia, thrombocytopenia (low platelet count), and/or neutropenia (low neutrophil count). Following such cancer treatment, patients are often unable to defend against infection. Supportive care for immunosuppression may include protective isolation of the patient such that the patient is not exposed to infectious agents; administration of: antibiotics, e.g., antiviral agents and antifungal agents; and/or periodic blood transfusions to treat anemia, thrombocytopenia and/or neutropenia.

A method of increasing the number of hematopoietic stem cells (HSC) by exposing a cell population comprising HSC to a c-myc antisense oligomer, in a manner effective to increase the number of hematopoietic stem cells for use in the treatment of a human cancer patient has been described in co-owned U.S. application Ser. No. 09/679,475 (PCT publication number, WO 01/25405), expressly incorporated by reference herein. The reference describes the use of c-myc treatment to increase the number of HSC and to increase the number of committed progenitor cells of particular lineages that derive from HSC, dependent upon culture conditions (WO 01/25405).

While the mechanism is not part of the invention, synergy between oncogenic pathways has been demonstrated previously and it has been suggested deregulation of c-myc expression selects for preferred secondary oncogenic pathways (D'Cruz et al., 2001). The results presented herein indicate that, although reduced levels of c-myc were achieved in antisense treated tumors, c-myc antisense oligomer treatment does not alter growth rates. This is consistent with a model in which c-myc expression influences the transformation process but is not the only factor involved in maintaining a transformed phenotype. The surprising and unexpected results observed following administration of an oligomer antisense to c-myc in a combination regimen with a traditional chemotherapeutic agent suggest that c-myc may also be important in maintaining the transformed phenotype. The results presented herein (Example 1) show that LLC1 tumors, which are inherently resistant to cisplatin, exhibit increased sensitivity to cisplatin in a treatment regimen that included an oligomer antisense to c-myc (AVI-4126, SEQ ID NO:1). Tumors were significantly more sensitive to cisplatin and taxol treatment and to a lesser extent etoposide when c-myc antisense oligomer treatment followed chemotherapy.

The methods described herein and related therapeutic regimens that combine traditional chemotherapy with administration of an oligomer antisense to c-myc also find utility in the treatment of polycystic kidney disease and in the treatment of cardiovascular disease. Of particular interest are treatment regimens that combine administration of cisplatin or taxol and administration of an oligomer antisense to c-myc. In such treatment regimens, the chemotherapeutic agent may be administered prior to, at the same time or following administration of the antisense oligomer.

B. Treatment Regimens

The present invention provides methods for cancer therapy, where an oligomer antisense to c-myc and one or more chemotherapeutic agents are administered to a patient. In a preferred aspect of the methods described herein, the c-myc antisense oligomer is administered to the patient prior to or following, but not at the same time as administration of the one or more chemotherapeutic agents.

In one preferred embodiment, cisplatin is administered to the patient prior to, or following, but not at the same time as, administration of the c-myc antisense oligomer.

In one exemplary embodiment, cisplatin (CDDP, Platinol), taxol (Paclitaxel) or etoposide (VP-16, VePesid Oral) is administered daily for 1 to 5 and preferably 3 consecutive days, followed by one or more days where no anti-cancer treatment is administered, then an oligomer antisense to c-myc is administered daily for 2 to 7 and preferably 5 consecutive days, with the cycle of chemotherapy and antisense oligomer administration repeated at least 2 times.

In another exemplary embodiment, cisplatin (cDDP, Platinol), taxol (Paclitaxel) or etoposide (VP-16, VePesid Oral) is administered daily for 1 to 5 and preferably 3 consecutive days, followed by administration of an oligomer antisense to c-myc daily for 2 to 7 and preferably 5 consecutive days, with the cycle of chemotherapy and antisense oligomer administration repeated at least 2 times.

In another preferred embodiment, the oligomer antisense to c-myc and chemotherapeutic agent are administered sequentially and at separate times spaced by at least one day. Preferably, the oligomer antisense to c-myc is administered daily for at least two days, followed by the administration of a chemotherapeutic agent for one or more days, with the cycle of alternating administration of the antisense oligomer to c-myc and the chemotherapeutic agent repeated at least two times. The time interval between administration of the two compounds is preferably at least three times the half-life of the last administered compound, to ensure that the last-administered compound is largely cleared from the patient before administration of the other compound. Typically, chemotherapeutic compounds are cleared with a half-life of 2-6 hours, so about 6-24 hours should be allowed for clearance. The oligomer antisense to c-myc is typically cleared with a half-life of 18-24 hours so a period of 2-3 days would be allowed for clearance.

As will be understood by those of skill in the art, the optimal treatment regimen will vary and it is within the scope of the treatment methods of the invention to evaluate the status of the disease under treatment and the general health of the patient prior to, and following one or more cycles of chemotherapy and antisense oligomer administration in order to determine if additional cycles of chemotherapy and antisense oligomer administration are indicated. Such evaluation is typically carried out by use of tests typically used to evaluate traditional cancer chemotherapy, as further described below in the section entitled “Monitoring Treatment”.

The preferred treatment regimens for use in practicing the invention generally include administration of the one or more chemotherapeutic agents prior to administration of a c-myc antisense oligomer. While the mechanism is not part of the invention, following chemotherapy a population of cancer cells that are refractory to the chemotherapy remain and such cells may be more differentiated and accordingly more susceptible to modification by a c-myc antisense oligomer that is administered following chemotherapy.

As detailed above, preferred antisense oligonucleotides for use in these methods are substantially uncharged phosphorodiamidate morpholino oligomers (PMOs), characterized by stability, high Tm, and capable of active or facilitated transport as evidenced by (i) competitive binding with a phosphorothioate antisense oligomer, and/or (ii) the ability to transport a detectable reporter into the cells.

In one preferred aspect of this embodiment, the oligomer is a PMO selected from the group consisting of the sequences presented as SEQ ID NO:1, SEQ ID NO:8, SEQ ID NO:9, SEQ ID NO:10, and SEQ ID NO:11.

C. Delivery of Chemotherapeutic Agents

An important aspect of the invention is effective delivery of one or more chemotherapeutic agents in a pharmaceutically acceptable carrier.

In accordance with one aspect of the invention, the choice of chemotherapeutic agent(s) and corresponding route and timing of delivery take advantage of one of more of: (i) established use in treatment of the particular type of cancer under treatment; (ii) the ability of the selected chemotherapeutic agent to result in an improved therapeutic when administered in combination with an oligomer antisense to c-myc; and (iii) local delivery of the chemotherapeutic agent by a mode of administration effective to achieve sufficient localized exposure of the agent to cancer cells.

In practicing the invention, the chemotherapeutic agent is administered by a route and using a treatment regimen that has an established use in cancer chemotherapy. As set forth above, the optimal route will vary with the chemotherapeutic agent. However, preferred routes typically include slow intravenous infusion (IV drip), oral administration and local injection. The formulations are easily administered in a variety of dosage forms such as injectable solutions, drug release capsules, implants or in combination with carriers such as liposomes or microcapsules.

Recommended dosages and dosage forms for a large number of chemotherapeutic agent have been established and can be obtained form conventional sources, such as the Physicians Desk Reference, published by Medical Economics Company, Inc., Oradell, N.J. If necessary, these parameters can be determined for each system by well-established procedures and analysis, e.g., in clinical trials.

For example, when orally administered, the active compounds may be combined with an inert diluent or in an edible carrier, or enclosed in hard or soft shell gelatin capsules, compressed into tablets, incorporated directly into food, incorporated with excipients and used in the form of ingestible tablets, buccal tables, troches, capsules, elixirs, suspensions, syrups, wafers, and the like. The appropriate amount of active compound is specific to the particular chemotherapeutic agent and is generally known in the art. The amount of active compound in such therapeutically useful compositions will be such that a suitable dosage is obtained.

Parenteral administration, may be accomplished using a suitable buffered aqueous solution and the liquid diluent which has been prepared in isotonic form using saline or glucose. Such aqueous solutions are appropriate for intravenous, intramuscular, subcutaneous and intraperitoneal administration. (See, for example, “Remington's Pharmaceutical Sciences”, 15th Edition, pages 1035-1038 and 1570-1580). Sterile injectable solutions are prepared by incorporating the chemotherapeutic agent in the required amount of an appropriate solvent with various other ingredients included, followed by filter sterilization. Sterile powders for use in sterile injectable solutions may be prepared by vacuum drying or freeze drying techniques or other means to result in a powder of the active i chemotherapeutic agent plus additional desired ingredients prepared from a previously sterile solution.

It will be understood that the invention contemplates treatment regimens that include the administration of one or more chemotherapeutic agents and administration of an oligomer antisense to c-myc for chemotherapy of cancer. Such a treatment regimen may be administered prior to, contemporaneously with, or subsequent to additional cancer treatment, such as radiation therapy, further chemotherapy and/or immunotherapy.

The present invention provides the advantage that the dose of the one or more chemotherapeutic agents may be decreased when administered in a treatment regimen that also includes c-myc antisense oligomer administration relative to treatment regimens that do not include -myc antisense oligomer administration. Such combination treatment are advantageous in patients that are young or old or whose cancer is recalcitrant to treatment regimens that do not include -myc antisense oligomer administration.

D. Delivery of Antisense Oligomers to the Patient

Effective delivery of an antisense oligomer to the target c-myc nucleic acid sequence is an important aspect of the methods of the invention. In accordance with one aspect of the invention, the modes of administration discussed below exploit one of more of the key features: (i) use of an antisense compound that has a high rate of cell uptake, (ii) the ability of the antisense compound to interfere with c-myc mRNA processing and mRNA translation, and (iii) delivery of the antisense oligomer by a mode of administration effective to achieve high localized concentration of the compound to cancer cells.

In accordance with the invention, effective delivery of an oligomer antisense to c-myc may include, but is not limited to, various systemic routes, including oral and parenteral routes, e.g., intravenous, subcutaneous, intraperitoneal, and intramuscular; as well as inhalation and transdermal delivery.

It is appreciated that any methods effective to deliver a c-myc antisense oligomer to into the bloodstream of a subject are also contemplated.

Transdermal delivery of antisense oligomers may be accomplished by use of a pharmaceutically acceptable carrier adapted for e.g., topical administration. One example of morpholino oligomer delivery is described in PCT patent application WO 97/40854, incorporated herein by reference.

The amount of the c-myc antisense oligonucleotide and the chemotherapeutic agent administered is such that the combination of the two types of agents is therapeutically effective. Dosages will vary in accordance with such factors as the age, health, sex, size and weight of the patient, the route of administration, the toxicity of the drugs, and the relative susceptibilities of the cancer to the oligonucleotide and chemotherapeutic agent.

Typically, one or more doses of antisense oligomer are administered, generally at regular intervals for a period of about one to two weeks. Preferred doses for oral administration are from about 1 mg oligomer/patient to about 25 mg oligomer/patient (based on an adult weight of 70 kg). In some cases, doses of greater than 25 mg oligomer/patient may be necessary. For IV administration, the preferred doses are from about 0.5 mg oligomer/patient to about 10 mg oligomer/patient (based on an adult weight of 70 kg). The antisense compound is generally administered in an amount sufficient to result in a peak blood concentration of at least 200400 nM antisense oligomer. Greater or lesser amounts of oligonucleotide may be administered as required and maintenance doses may be lower.

In general, the method comprises administering to a subject, in a suitable pharmaceutical carrier, an amount of the antisense agent effective to inhibit expression of the c-myc nucleic acid target sequence.

It follows that the antisense oligonucleotide composition may be administered in any convenient vehicle, which is physiologically acceptable. Such an oligonucleotide composition may include any of a variety of standard physiologically acceptable carriers employed by those of ordinary skill in the art. Examples of such pharmaceutical carriers include, but are not limited to, saline, phosphate buffered saline (PBS), water, aqueous ethanol, emulsions such as oil/water emulsions, triglyceride emulsions, wetting agents, tablets and capsules. It will be understood that the choice of suitable physiologically acceptable carrier will vary dependent upon the chosen mode of administration. In some instances liposomes may be employed to facilitate uptake of the antisense oligonucleotide into cells. (See, e.g., Williams A S, 1996; Lappalainen et al., 1994; Nakamura et al., 2001; and Lou et al., 2001.) Hydrogels may also be used as vehicles for antisense oligomer administration, for example, as described in WO 93/01286. Alternatively, the oligonucleotides may be administered in microspheres or microparticles. (See, e.g., Wu et al., 1999.) Sustained release compositions are also contemplated within the scope of this application. These may include semipermeable polymeric matrices in the form of shaped articles such as films or microcapsules.

It will be understood that the effective in vivo dose of a c-myc antisense oligonucleotide for use in the methods of the invention will vary according to the frequency and route of administration as well as the condition of the subject under treatment. Accordingly, such in vivo therapy will generally require monitoring by tests appropriate to the condition being treated and a corresponding adjustment in the dose or treatment regimen in order to achieve an optimal therapeutic outcome.

In one preferred embodiment, the oligomer is a phosphorodiamidate morpholino oligomer (PMO), contained in a pharmaceutically acceptable carrier, and delivered orally. In a further aspect of this embodiment, a morpholino c-myc antisense oligonucleotide is administered at regular intervals for a short time period, e.g., daily for two weeks or less. However, in some cases the antisense oligomer is administered intermittently over a longer period of time.

In some cases, the treatment regimen will include further intervention such as radiation therapy, immunotherapy and/or additional chemotherapy. Such treatment may occur prior to, during or subsequent to administration of the chemotherapeutic agent and c-myc antisense oligomer.

VI. Evaluating the Effect of Antisense Oligomers

A. Analysis of the Effects of Antisense Oligomer Treatment

Candidate antisense oligomers are evaluated, according to well known methods, for acute and chronic cellular toxicity, such as the effect on protein and DNA synthesis as measured via incorporation of ³H-leucine and ³H-thymidine, respectively. In addition, various control oligonucleotides, e.g., control oligonucleotides such as sense, nonsense or scrambled antisense sequences, or sequences containing mismatched bases, in order to confirm the specificity of binding of candidate antisense oligomers. The outcome of such tests are important to discern specific effects of antisense inhibition of gene expression from indiscriminate suppression. (See, e.g. Bennett et al., 1995). Accordingly, sequences may be modified as needed to limit non-specific binding of antisense oligomers to non-target sequences.

The effectiveness of a given antisense oligomer molecule in forming a heteroduplex with the target RNA may be determined by screening methods known in the art. For example, the oligomer is incubated a cell culture expressing c-myc, and the effect on the target RNA is evaluated by monitoring the presence or absence of (1) heteroduplex formation with the target sequence and non-target sequences using procedures known to those of skill in the art, (2) the amount of c-myc mRNA, as determined by standard techniques such as RT-PCR or Northern blot, or (3) the amount of c-myc protein, as determined by standard techniques such as ELISA or Western blot.

B. Animal Models

An animal model routinely employed by those of skill in the art to evaluate anti-cancer therapies was used to demonstrate the efficacy of the methods of the present invention. Examples are described in Smith et al., 2000. The model is based on the transplantation of Lewis lung cells (LLC1), a highly tumorigenic lung carcinoma cell line, into syngeneic C57-bik mice. LLC1 produce discernable tumors by day 4 when 200,000 cells are transplanted subcutaneously onto the right flanks of C57-BL mice. Therapeutic efficacy was evaluated based on daily caliper measurements of tumor length and width and tumor weights at the end of 25 day studies. A 20 base PMO antisense to c-myc mRNA (AVI-4126, SEQ ID NO:1) was evaluated for efficacy in the model. Intact AVI-4126 was found in tumor tissue following ip administration at 300 μg/mouse/day which diminished c-myc expression but failed to significantly reduce tumor growth. AVI-4126 was also administered i.p. (300 μg/mouse/day) in combination with chemotherapy. Co-administration of cisplatin (83 μg/mouse/day) on days 2-4 and 13-15 with AVI-4126 on days 2-8 and 13-19 had no additional effect on tumor growth inhibition versus cisplatin alone (Example 1). A combination regimen in which cisplatin was administered on days 2-4 and 13-15 followed by AVI-4126 on days 6-12 and 17-23 inhibited tumor growth significantly more than cisplatin alone indicating that the anti-tumor effect requires a dosing schedule which separates cisplatin and AVI-4126 treatments (FIG. 8A). This increase in anti-tumor activity was demonstrated in combination with taxol and to a lesser extent with etoposide.

The results further described in Example 1 illustrate that treatment with AVI-4126 inhibits expression of c-myc in LLC1 tumors and has potential as a potent anti-cancer agent in combination chemotherapy.

VII. Monitoring Treatment

The efficacy of a given therapeutic regimen involving the methods described herein, may be monitored, e.g., using diagnostic techniques appropriate to the type of cancer under treatment.

The exact nature of an evaluation will vary dependent upon the condition being treated and the treatment regimen may be adjusted (dose, frequency, route, etc.), as indicated, based on the results of such diagnostic tests.

It will be understood that an effective in vivo treatment regimen using the antisense oligonucleotides of the invention will vary according to the frequency and route of administration, as well as the condition of the subject under treatment (i.e., prophylactic administration versus administration in response to localized or systemic infection). Accordingly, such in vivo therapy will generally require monitoring by tests appropriate to the particular type of condition, e.g., cancer, under treatment and a corresponding adjustment in the dose or treatment regimen in order to achieve an optimal therapeutic outcome.

Diagnosis and monitoring of cancer generally involves one or more of (1) biopsy, (2) ultrasound, (3) x-ray, (4) magnetic resonance imaging, (5) nucleic acid detection methods, (6) serological detection methods, i.e., conventional immunoassay and (7) other biochemical methods. Such methods may be qualitative or quantitative.

The efficacy of a given therapeutic regimen involving the methods described herein may be monitored, e.g., by general indicators of the disease condition under treatment, as further described above.

Nucleic acid probes may be designed based on c-myc or other nucleic acid sequences associated with the particular cancer under treatment. Nucleic amplification tests (e.g., PCR) may also be used in such detection methods.

It will be understood that the exact nature of diagnostic tests as well as other physiological factors indicative of a disease condition will vary dependent upon the particular condition being treated and whether the treatment is prophylactic or therapeutic.

In cases where the subject has been diagnosed as having a particular type of cancer, the status of the cancer is also monitored using diagnostic techniques typically used by those of skill in the art to monitor the particular type of cancer under treatment.

The antisense oligomer treatment regimen may be adjusted (dose, frequency, route, etc.), as indicated, based on the results of immunoassays, other biochemical tests and physiological examination of the subject under treatment.

VIII. Applications/Utility of the Invention

As described herein, treatment of cancer with a c-myc antisense oligonucleotide in combination with traditional cancer treatment such as chemotherapy and/or radiation therapy find utility in slowing or eliminating the growth and/or spread of the cancer. For example, the methods of the invention can: (1) inhibit or arrest the growth of cancer cells; (2) allow for lower dose and/or shorter term administration of chemotherapeutic agents resulting in a decrease in toxic side effects; (3) allow for lower dose or shorter term administration of chemotherapeutic agents decreasing the likelihood of development of resistance to the chemotherapeutic agent; (4) provide a type of antisense oligomer (e.g., a PMO) that is substantially uncharged and does not coprecipitate with the chemotherapeutic agent; and (5) provide an alternative and efficacious treatment regimen for patient populations that cannot tolerate doses of a chemotherapeutic agent required for efficacy when administered in a treatment regimen that lacks c-myc antisense oligomer administration.

All patent and literature references cited in the present specification are hereby incorporated by reference in their entirety.

The following examples illustrate but are not intended in any way to limit the invention.

Materials and Methods

Morpholino oligomer synthesis. Morpholino phosphorodiamidate oligomers (PMOs) with sequence complementary to the c-myc translation start site (AVI-4126; SEQ ID NO:1), a mouse p21 sequence (SEQ ID NO:3), a mouse RAD51 sequence (SEQ ID NO:4) and a scrambled control (SEQ ID NO:2), were synthesized and purified by AVI BioPharma, Inc. (Corvallis, Oreg.). Purity was greater than 90% as determined by reverse phase HPLC and MALDI TOF mass spectrometry. Lyophilized PMOs were dissolved in sterile saline for injection.

Tumor cells. The Lewis lung cell line (LLC1) used in the studies described herein was derived from the Lewis lung carcinoma established in 1951 by Dr. M. R. Lewis and has been utilized for the evaluation of cancer chemotherapeutic agents Mayo, 1972; Bertram et al., 1980). Lewis lung carcinoma cells (ATCC, Manassas, Va.) were maintained in DMEF-12 medium supplemented with 10% fetal bovine serum, penicillin (100 units/mL), streptomycin (100 μg/mL), and amphotericin (0.25 μg/mL) at 37° C. in a 5% CO₂/95% air humidified incubator. Cells were harvested as an approximately 70% confluent culture of log growth phase at the time of transplant and were injected as a cell suspension in media at a concentration of 200,000 cells per 100 μl injection.

Syngeneic mice. C57BL/6J mice (Simonsen, Gilroy, Calif.) weighing 22 to 24 g were housed in sterile plastic cages at the Laboratory Animal Resources Facility at Oregon State University (OSU), Corvallis, Oreg. Mice were given access to rodent chow (Harlan Teklad, Madison, Wis.) and tap water ad libitum and exposed to 12 hour light/dark cycles. All animal protocols conformed to the ethical guidelines of the 1975 Declaration of Helsinki and were approved by the ‘Institutional Animal Care and Use Committee’ of OSU.

Immunoblot analysis of c-myc protein. All antibodies were obtained from Santa Cruz Biotechnology (Santa Cruz, Calif.). Three-hundred micrograms of LLC1 protein lysate was analyzed on a 5% SDS/acrylamide stacking gel with a 12% v/v sodium dodecylsulfate (SDS)/acrylamide separating gel. Gels were blotted, probed, and visualized according to standard Western blotting protocols. Membranes were probed with rabbit anti-mouse c-myc polyclonal antibodies N-262 (sc-764) or C-19 (sc-788) diluted 1:2000 in blocking buffer (Genotech) followed by goat-anti rabbit HRP-conjugated antibody (sc-2054). The relative amount of c-myc and actin protein was visualized by ECLplus (Amersham, Piscataway, N.J.) and analyzed on a Kodak 440 Image Station using 1 D Image Analysis software (Kodak, Rochester N.Y.). Membranes were then soaked in stripping buffer (Genotech) for 20 minutes at 25° C. and reprobed with a 1:2000 dilution of goat anti-mouse β-actin polyclonal antibody (sc-1616), which served as a protein loading control, followed by donkey anti-goat HRP-conjugated antibody (sc-2056).

HPLC detection of AVI-4126 in LLC1 tumor tissue. The presence of AVI-4126 in tumor tissue lysates (prepared 4 hours after a single injection of 100 μg of AVI-4126 into tumor bearing mice) was determined via a 5′-flouresceinyl DNA:AVI-4126 (FDNA:AVI-4126) duplex detection method developed at AVI BioPharma (Corvallis, Oreg.). Briefly, 500 ng of internal standard (10 μl of a 2.29 mg PMO/mL of 0.025M Tris buffer, pH=8) of a 15mer internal PMO standard complementary to the FDNA probe (5′-GAGGGGCATCGTCGC-3′ (SEQ ID NO: 14)) was added to each tumor tissue lysate. The lysate was combined with 250 μl methanol and mixed thoroughly. The sample was centrifuged 10 minutes at 15,000× g and the supernatant was removed and heated to 70° C. for g 10 minutes. The sample was centrifuged for 10 minutes and the supernatant was dried down in a Savant SC110 speed vacuum at low heat for 1 hour. The dried sample was then combined with 100 μl Tris buffer in clear shell vials and lyophilized. Each lyophilized sample was rehydrated with a 100 μl aliquot of a 1.0 OD/ml 5′-fluoresceinyl DNA probe (FDNA, 5′-fluoresceinyl-GCGACGATGCCCCTCAACGT-3′ (SEQ ID NO: 15)) with sequence complementary to AVI-4126.

The entire 100 μl sample was then analyzed for the presence of FDNA:AVI-4126 duplex using reverse phase HPLC with fluorescence detection. The sample was injected into a Dionex DNAPac PA-100 column (4×250) using a Varian HPLC pump (model 9010 inert) equipped with a fluorescence detector and Al-200 autosampler (100 μl injector loop volume). The mobile phases (A: 0.025M Tris, pH=8 and B: 0.025M Tris/1 M NaCl pH=8) were prepared using HPLC grade solvents filtered through a 0.2 micron filter prior to use. The pump gradient program was 90% A+10% B (0 min) and 55% A+45% B (20 min.) at a flow rate of 1.5 ml/min with fluorescence detection at a 494 nm (excitation) and 518 nm emission wavelengths.

ICP-MS Detection and Quantitation of platinum/cisplatin. A 200 μL aliquot of tissue lysate (40 mg of LLC1 tumor tissue) was dissolved in 1.33 mL of aqua regia followed by a 10 fold dilution. The samples were then analyzed by ICP-MS technique for the presence of Pt according to the method of Long et al (16) by Anatek Labs (Moscow, ID).

Statistical Analysis. All data are reported as the mean ±SEM were determined by the computer program InStat2 (GraphPad, San Diego). The p values were calculated by InStat2 using ANOVA and the Tukey multiple comparison test. Graphs, linear regression, and slopes were generated using Prism v2.0 (GraphPad).

### EXAMPLE 1

Tumor studies with AVI-4126 and Chemotherapy Following a 5 day acclimation period, C57BL/6J mice (Simonsen, Gilroy, CA) cared for as set forth above, were anesthetized with isoflurane, shaved, and injected subcutaneously in the right rear flank with approximately 200,000 viable LLC1 cells (study day 0). Injection sites were monitored daily to ensure that solid, homogeneous tumor growth was consistently obtained 4 days after LLC1 cell injection. Chemotherapy injections were prepared fresh daily before i.p injection (see Table 1). All PMO's and cisplatin (Sigma, St. Louis, Mo.) were dissolved in sterile, apyrogenic saline (Sigma) adjusted to an injection volume of 0.1 ml. A Taxol stock solution (6 mg/ml in Cremophore EL and ethanol, Bristol Myers Squibb, Syracuse, N.Y.) was diluted to 1 mg/ml in 1× PBS prior to injection. Etoposide (Sigma) stock solutions were prepared by dissolving in 70% ethanol at 11 mg/ml followed by dilution with saline to a final concentration of 5 mg/ml. 5-FU (Calbiochem) was dissolved in saline at a concentration of 12.5 mg/ml.

Morpholino phosphorodiamidate oligomers (PMOs) with a sequence complementary to the c-myc translation start site (AVI-4126; SEQ ID NO:1), a mouse p21 sequence (SEQ ID NO:3), a mouse RAD51 sequence (SEQ ID NO:4) and a scrambled control (SEQ ID NO:2), were synthesized and purified as set forth above.

An initial study was performed to determine AVI-4126 levels in the tumor and to evaluate sequence specific inhibition of c-myc protein levels. Tumor bearing mice 24 days post LLC1 transplant were given injections of either saline, AVI-4126 or scrambled control (100 μg/mouse IP). Tumors were excised 4 hours later and evaluated for AVI-4126 and analyzed by immunoblot for c-myc protein, as described below.

The treatment groups employed in three studies (A, B and C) are summarized in Table 1. Animals were treated as described. Therapeutic efficacy was evaluated based on daily measurement of tumor area (length x width, cm²) with digital calipers and tumor mass determined at the time of euthanization. Mice in all studies were euthanized by asphyxiation with CO₂ on the final day of PMO treatments. Tumors were immediately removed, weighed and approximately 0.25 g tumor tissue was homogenized in 1.0 ml Tissue-PE lysis buffer (Genotech, St. Louis, Mo.) containing protease inhibitor cocktail tablets (Complete™ Mini EDTA-free, Boehringer-Mannheim) which were dissolved in the lysis buffer 30 minutes before tissue homogenization. Lysates were centrifuged at 15,000× g for 15 minutes at 4° C. and 150 μl aliquots of supernatant were combined 1:1 with electrophoresis sample buffer and boiled at 100° C. for 5 minutes.

TABLE 2
Combination Chemotherapy Regimens

| STUDY | TREATMENT |
|---|---|
| A | (1) Saline |
| | (2) Cisplatin (83 μg/mouse/day IP) days 2-4, 14-16. |
| | (3) AVI-4126 (300 μg/mouse/day IP) days 2-8, 14-21. |
| | (4) Cisplatin (83 μg/mouse/day IP) days 2-4, 14-16 |
| | and |
| | AVI-4126 (300 μg/mouse/day IP) days 6-12, 18-23. |
| | (5) Cisplatin (83 μg/mouse/day IP) days 2-4, 14-16 |
| | and |
| | AVI-4126 (300 μg/mouse/day IP) days 2-8, 13-19 |
| B | (1) Saline |
| | (2) Etoposide (375 μg/mouse/day IP) days 2-4, 14-16. |
| | (3) Etoposide (375 μg/mouse/day IP) days 2-4, 14-16 |
| | and |
| | AVI-4126 (300 μg/mouse/day IP) days 6-12, 18-23 |
| C | (1) Saline |
| | (2) Taxol (125 μg/mouse/day IP) days 2-4, 14-16. |
| | (3) Taxol (125 μg/mouse/day IP) days 2-4, 14-16 and |
| | AVI-4126 (300 μg/mouse/day IP) days 6-12, 18-23 |
| D | (1) Saline |
| | (2) 5-FU (1250 μg/mouse/day IP) days 2-4, 14-16. |
| | (3) 5-FU (1250 μg/mouse/day IP) days 2-4, 14-16 and |
| | AVI-4126 (300 μg/mouse/day IP) days 6-12, 18-23 |

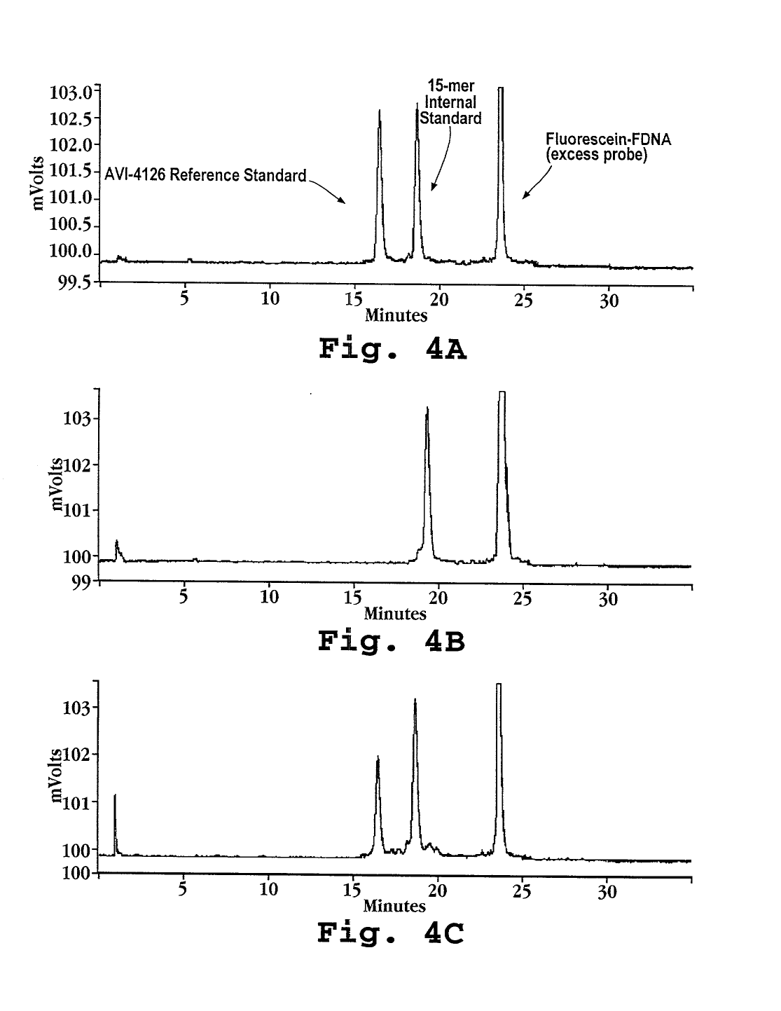

A. AVI4126 is detectable in LLC1 tumor tissue. HPLC fluorescence detection of AVI4126 was performed in tumor tissue lysates from mice treated with AVI-4126 or saline. A representative HPLC analysis showing a fluorescence peak representing FDNA:AVI-4126 duplex was readily detectable only in mice treated with AVI-4126 (FIG. 4C). No evidence of degradation of AVI-4126 was observed. Administration of AVI-4126 did not effect platinum levels in the tumor tissue (data not shown).

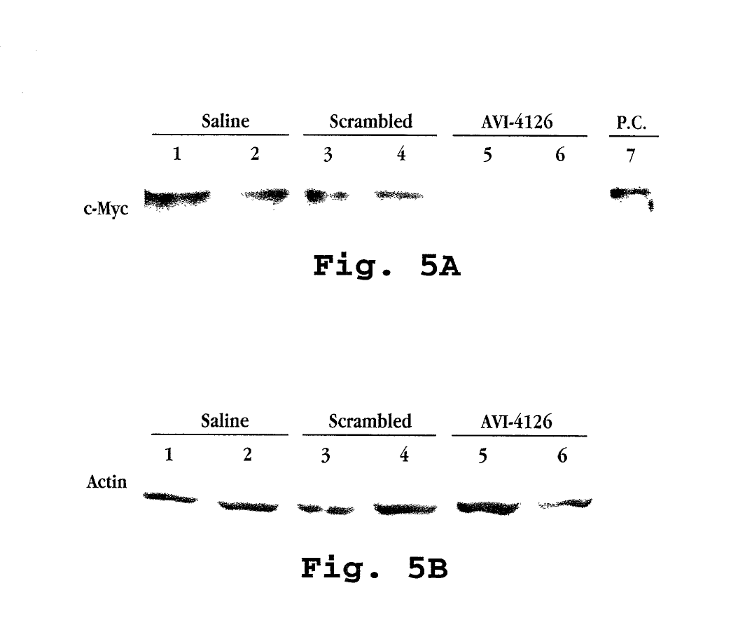

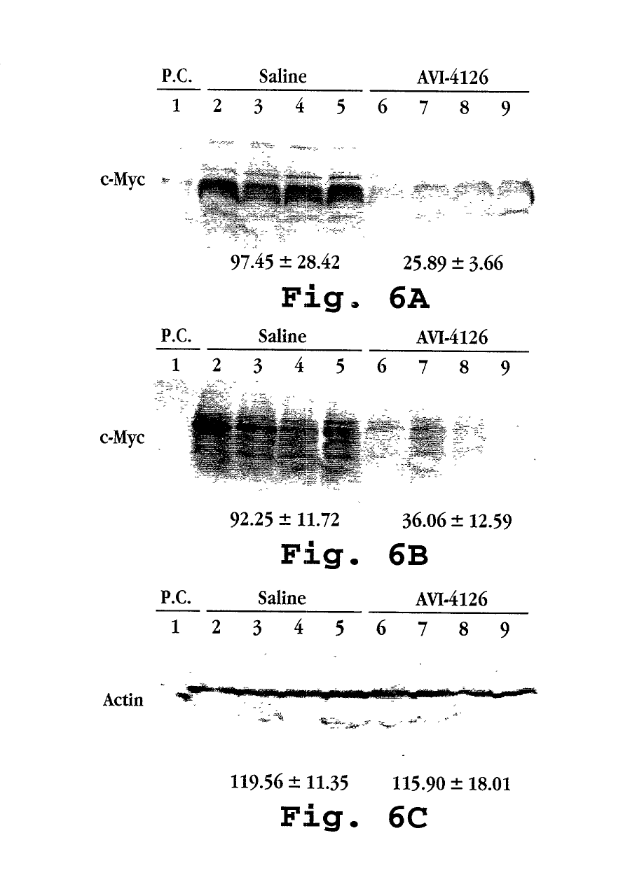

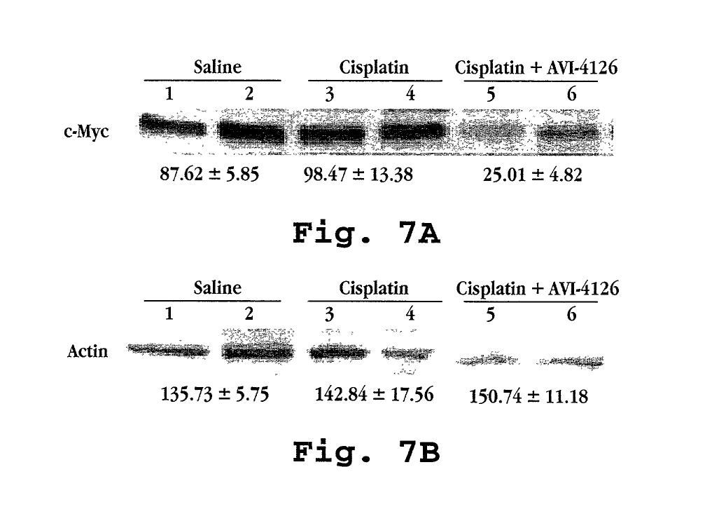

B. AVI-4126 reduces c-myc levels in LLC1 tumor tissue. Immunoblot analysis was performed to determine c-myc levels in tumor tissue. A single injection of AVI-4126 reduced levels of c-myc by 77% and 63% relative to levels detected in saline and scrambled PMO controls (FIG. 5A). c-myc was similarly reduced relative to controls in lysates from tumors harvested from mice treated with saline, AVI-4126 alone, cisplatin, or a combination of AVI-4126 and cisplatin as described in Table 2A, group (5). (See FIGS. 7 and 8A.) Animals treated AVI-4126 and cisplatin in which the dose was separated did not yield sufficient tumor tissue to perform immunoblot analysis. Four tumors from representative animals in the saline or AVI-4126 treatment groups are presented in FIGS. 6A and B which show images of immunoblots probed with n-terminal and c-terminal specific c-myc antibodies. Analysis of band intensity normalized to β-actin protein levels (FIG. 6C) reveals a 74% (n-terminal antibody, FIG. 6A) and 61% (c-terminal antibody, FIG. 6B) inhibition c-myc levels compared to saline control in representative tumors from animals treated with saline or AVI-4126 alone. Bands appear at approximately 66 kD with no evidence of 38 kD bands that have been reported for c-myc splice variants in human cells caused by AVI-4126 (13). Analysis of immunoblots presented in FIG. 7A reveals that mice administered cisplatin +AVI-4126 have a reduction in c-myc (72% compared to saline). There was no statistical difference between c-myc levels in cisplatin treated groups compared to saline.

C. Antitumor effects of combination chemotherapy with AVI-4126 and cisplatin is schedule dependent. The daily tumor area for all treatment groups is presented in FIGS. 8A-C. Tumor growth was analyzed in a combination study in which cisplatin was administered alone, co-administered with AVI-4126 or administered in an alternating regimen with AVI-4126 treatment. When tumor bearing mice were given two rounds of treatment in which AVI-4126 was co-administered with cisplatin (see Table 2A, group 5), the tumor growth rate and mass at the end of the study was no different from cisplatin treatment alone (data not shown). However, tumor growth rates in groups which received cisplatin were significantly lower than groups treated with AVI-4126 alone or saline (p<001).

When tumor bearing mice were administered two rounds of a regimen which staggered the administration of cisplatin and AVI-4126 (Table 2A, group 4), there was a significant reduction in tumor growth rate compared to mice administered two rounds of cisplatin alone (p<001) or saline (p<001) (FIG. 8 panel A and Table 3). Necropsies of tumors from the regimen in which cisplatin and AVI-4126 treatment was staggered revealed very small, non-invasive tumor nodules while tumors in the cisplatin alone and cisplatin plus co-administered AVI-4126 treated mice were highly invasive and vascularized.

Additional studies which staggered the administration of etoposide, taxol or 5-FU with AVI-4126 revealed that enhanced efficacy depends on the chemotherapy. Cisplatin is most effective followed by etoposide and taxol. The efficacy of all three agents were significantly enhanced by addition of AVI-4126 to the treatment regimen (p<001 for all three treatments compared to respective single agent treatment regimens or saline as indicated by TGR. 5-FU was relatively ineffective as a single agent or combined with AVI-4126 (See Table 3 and FIG. 8D).

Control PMO oligomers for AVI-4126 have been extensively studied and previously reported (Hudziak et al., 2000).

The scrambled control PMO (SEQ ID NO:2) had no effect on c-myc levels. To test a PMO backbone in the same protocol as AVI-4126, two PMOs were utilized, p21AS (SEQ ID NO:3) and RAD51AS (SEQ ID NO:4) were synthesized as described above and tested in this model utilizing the same regimen that was effective with AVI-4126. These sequences failed to produce enhanced effects when combined with cisplatin. Tumor growth rates and mass at the end of the studies for these molecules are shown in Table 3.

In summary, the results shown in Table 3 demonstrate that a treatment protocol where a c-myc antisense oligomer is administered in an alternating regimen with either cisplatin, etoposide, or taxol, resulted in a significant reduction in tumor growth rate (TGR) and tumor size, 18.6%, 36.8%, 50.9%, respectively.

TABLE 3
Summary of tumor mass and growth rate for various combination
treatments

| | | | | % of |
|---|---|---|---|---|
| | | Tumor Mass | Tumor Growth Rate* | Saline |
| Treatment | n | (gm ± STE) | (TGR) | TGR |
| saline | 29 | 1.508 ± 0.181 | 0.204 ± 0.014 | 100.0 |
| AVI-4126 | 6 | 1.788 ± 0.651 | 0.212 ± 0.031 | 103.9 |
| cisplatin | 26 | 0.550 ± 0.081 | 0.101 ± 0.011 | 49.5 |
| cisplatin + AVI- | 6 | 0.112 ± 0.059 | 0.038 ± 0.011 | 18.6 |
| 4126 | | | | |
| p21AS | 6 | 1.260 ± 0.389 | 0.184 ± 0.030 | 90.2 |
| cisplatin + | 6 | 0.607 ± | 0.108 ± 0.018 | 50.9 |
| p21AS | | 0.194 | | |
| RAD51AS | 6 | 1.193 ± 0.166 | 0.198 ± 0.017 | 97.1 |
| cisplatin + | 6 | 0.322 ± 0.076 | 0.089 ± 0.012 | 43.6 |
| RAD51AS | | | | |
| etoposide | 5 | 0.970 ± 0.244 | 0.153 ± 0.025 | 75.0 |
| etoposide + AVI- | 5 | 0.560 ± 0.258 | 0.075 ± 0.016 | 36.8 |
| 4126 | | | | |
| Taxol | 6 | 1.827 ± 0.210 | 0.234 ± 0.023 | 114.7 |
| Taxol + AVI- | 6 | 0.558 ± 0.279 | 0.104 ± 0.022 | 50.9 |
| 4126 | | | | |
| 5-FU | 6 | 2.720 ± 0.991 | 0.234 ± 0.044 | 114.7 |
| 5-FU + AVI-4126 | 6 | 1.018 ± 0.224 | 0.180 ± 0.018 | 88.2 |

*Tumor growth rate is calculated using linear regression to analyze the change tumor area from day 14 to 25 when the change in tumor area is linear. The data presented is the slope ± standard deviation.

TABLE 4

Sequences Provided In Support of the Invention

| | |
|---|---|
| | SEQ |
| | ID |
| Description | NO |

| | |
|---|---|
| antisense to c-myc AUG; AVI 4126: ACG TTG AGG | 1 |
| GGC ATC GTC GC | |
| c-myc antisense scramble control AVI 4144: ACT | 2 |
| GTG AGG GCG ATC GCT GC | |
| c-myc antisense control:mouse p21: CAT CAC CAG | 3 |
| GAT TGG ACA TGG | |
| c-myc antisense control:mouse RAD51: CAA GCT | 4 |
| GCA TTT GCA TAG CCA T | |
| antisense to c-myc, AVI #92: GMT MMM TMT GTM | 5 |
| TMT MGM TGG | |
| antisense to c-myc, AVI #93: MMG MMM GMT MGM | 6 |
| TMM MTM TG | |
| antisense to c-myc, AVI #25: GGC AUC GUC GUG | 7 |
| ACU GUC GGG UUU UCC ACC | |
| antisense to c-myc, AVI #21: GGG GCA UCG UCG | 8 |
| UGA CUG UCU GUU GGA GGG | |
| antisense to c-myc, AVI #108: CGU CGU GAC UGU | 9 |
| CUG UUG GAG | |
| antisense to c-myc, AVI #111: CGT CGT GAC TGT | 10 |
| CTG TTG GAG G | |
| antisense to c-myc, AVI #37: GGC AUC GUC GCG | 11 |
| GGA GGC UGC UGG AGC G | |
| antisense to c-myc, AVI #26: CCG CGA CAU AGG | 12 |
| ACG GAG AGC AGA GCC C | |
| antisense to c-myc, AVI 4174: TTG AGG GGC ATC | 13 |

It is claimed:

1. In an improved method for the treatment of cancer susceptible to treatment by chemotherapy, the improvement comprising:
administering an oligomer antisense to c-myc to a cancer patient in combination with a chemotherapeutic agent, wherein said oligomer antisense to c-myc and said chemotherapeutic agent is to be administered sequentially at spaced apart time intervals of several hours after administration of the chemotherapeutic agent and at least one day after administration of the oligomer antisense.

2. The method of claim 1, wherein the oligomer antisense to c-myc is between 12-25 bases in length and contains the sequence selected from the group consisting of SEQ ID NO:1, SEQ ID NO:8, SEQ ID NO:9, SEQ ID NO:10, and SEQ ID NO:11.

3. The method of claim 1, wherein the antisense oligomer is characterized by,
(a) a backbone which is substantially uncharged;
(b) the ability to hybridize with the complementary sequence of a target RNA with high affinity at a Tm greater than 50° C.;
(c) nuclease resistance; and
(d) the capability for active or facilitated transport into cells.

4. The method of claim 3, wherein the antisense oligomer backbone has a selected from the group consisting of

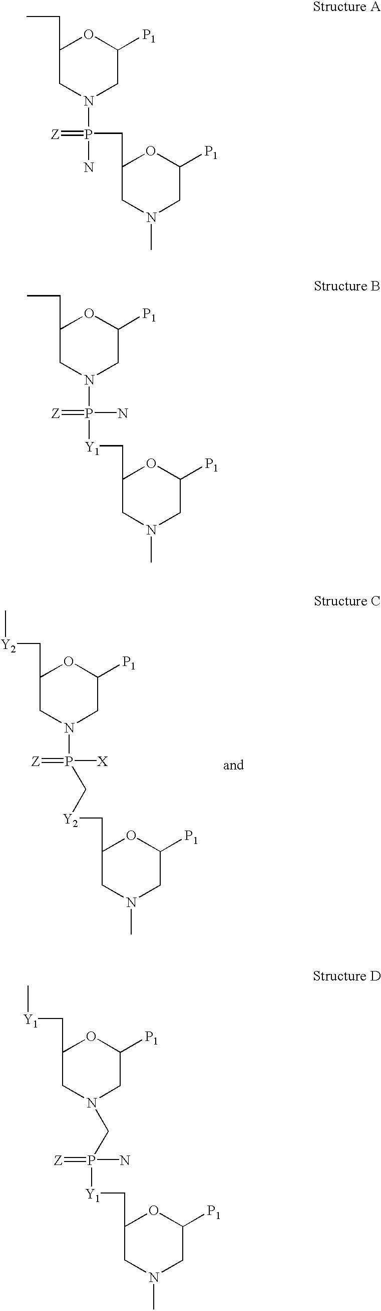

5. The method of claim 1, wherein said chemotherapeutic agent is selected from the group consisting of cisplatin, etoposide (VP-16), taxol, and analogs and derivatives thereof.

6. The method of claim 1, wherein administering of the antisense oligomer to c-myc begins at least one day after administering said chemotherapeutic agent.

7. The method of claim 6, wherein the administration of said oligomer composition, followed at least one day later by the administration of the chemotherapeutic agent represents a cycle of therapy which is repeated multiple times, each cycle separated by at least one day.

8. A kit for the treatment of cancer susceptible to treatment by chemotherapy, comprising a first composition comprising an oligomer antisense to c-myc and a second composition comprising a chemotherapeutic agent, wherein the first composition and the second composition are to be administered sequentially at spaced apart time intervals of at least one day after administration of the first composition and several hours after administration of the second composition.

9. The kit of claim 8, wherein the oligomer antisense to c-myc is between 12-25 bases in length and contains the sequence selected from the group consisting of SEQ ID NO:1, SEQ ID NO:8, SEQ ID NO:9, SEQ ID NO:10, and SEQ ID NO:11.

10. The kit of claim 8, wherein the antisense oligomer is characterized by,
(a) a backbone which is substantially uncharged;
(b) the ability to hybridize with the complementary sequence of a target RNA with high affinity at a Tm greater than 50° C.;
(c) nuclease resistance; and
(d) the capability for active or facilitated transport into cells.

11. The kit of claim 10, wherein the antisense oligomer backbone has a structure selected from the group consisting of

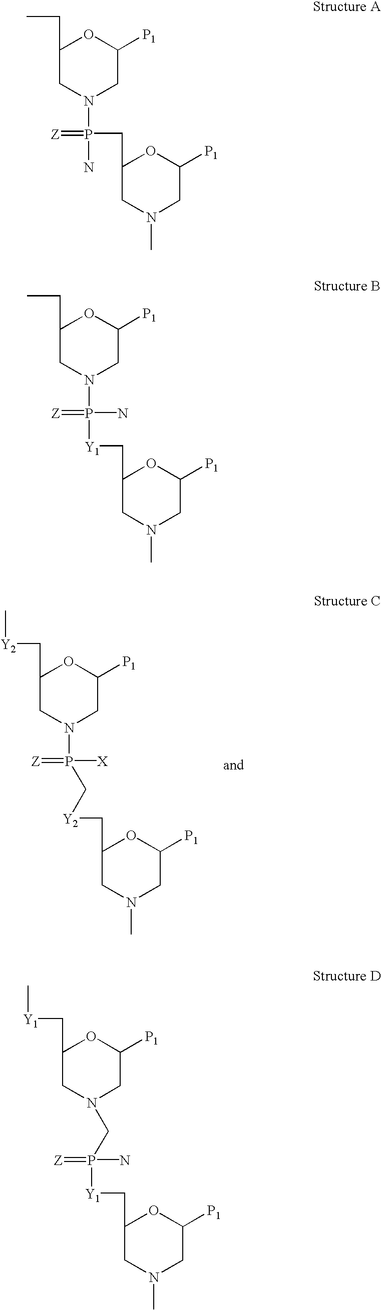

12. The kit of claim 8, wherein said chemotherapeutic agent is selected from the group consisting of cisplatin, etoposide (VP-16), taxol, and analogs and derivatives thereof.

13 The kit of claim 8, wherein administering of the antisense oligomer to c-myc begins at least one day after administering said chemotherapeutic agent.

14. The kit of claim 13, wherein the administration of said oligomer composition, followed at least one day later by the administration of the chemotherapeutic agent represents a cycle of therapy which is repeated multiple times, each cycle separated by at least one day.

15. An oligomer composition for the treatment of cancer in a patient currently being treated by chemotherapy, comprising an oligomer antisense to c-myc, wherein the composition is administered prior to or following administration of a chemotherapeutic agent.

16. The oligomer composition of claim 15, which is between 12-25 bases in length and contains the sequence selected from the group consisting of SEQ ID NO:1, SEQ ID NO:8, SEQ ID NO:9, SEQ ID NO:10, and SEQ ID NO:11.

17. The oligomer of claim 15, which is characterized by,
(a) a backbone which is substantially uncharged;
(b) the ability to hybridize with the complementary sequence of a target RNA with high affinity at a Tm greater than 50° C.;
(c) nuclease resistance; and
(d) the capability for active or facilitated transport into cells.

18. The oligomer composition of claim 17, which has a structure selected from the group consisting of [chemical structure]
